(19) United States
(12) Patent Application Publication
(10) Pub. No.: US 2011/0020457 A1
(43) Pub. Date: Jan. 27, 2011

(54) POLYMER-SURFACTANT NANOPARTICLES FOR SUSTAINED RELEASE OF COMPOUNDS

(75) Inventors: Jayanth Panyam, Novi, MI (US); Mahesh D. Chavanpatil, Detroit, MI (US)

Correspondence Address: Douglas Gergich;Intellectual Property Docketing Department, 925 Fourth Avenue, Suite 2900, Seattle, WA, 98104-1158, US

(73) Assignee: Wayne State University

(21) Appl. No.: 12/377,597

(22) PCT Filed: Aug. 14, 2007
(86) PCT No.: PCT/US07/75925

§ 371 Date: Oct. 13, 2010

Related U.S. Application Data
(60) Provisional application No. 60/837,808, filed on Aug. 14, 2006.

(51) Int. Cl. A61K 9/14; A61K 31/704; A61K 47/36; A61K 31/137; A61K 31/4168; A61K 31/122; A61K 31/56; A61K 31/60; A61K 31/519; A61K 38/13; A61K 31/4436; A61K 31/573; A61K 31/047; A61P 17/06; A61P 35/00

(52) U.S. Cl. 424/499; 514/34; 514/779; 514/654; 514/398; 514/680; 514/169; 514/161; 514/249; 514/18.7; 514/337; 514/179; 514/729; 977/773; 977/915

(57) ABSTRACT

A polymer-surfactant nanoparticle formulation, using the anionic surfactant aerosol OT (AOT) and polysaccharide polymer alginate, is used for sustained release of water-soluble drugs. The AOT-alginate nanoparticles are suitable for encapsulating doxorubicin, verapamil and clonidine, as well as therapeutic agents effective against dermal conditions such as psoriasis. The nanoparticles are also suitable for encapsulating photo-activated compounds such as methylene blue for use in photo-dynamic therapy of cancer and other diseases, and for treating tumor cells that exhibit resistance to at least one chemotherapeutic drug.

### FIELD OF THE INVENTION

The present invention relates to compositions and methods useful for sustained release of drugs or therapeutic agents.

### BACKGROUND

Many clinically important small molecular weight drugs including anticancer agents (Binaschi, M. et al., Curr Med Chem Anti-Canc Agents 1:113-130, 2001; Zhao, J. et al., Int J Oncol 27:247-256, 2005), corticosteroids (Adcock, I. M. and Ito, K., Proc Am Thorac Soc 2, 313-319, 2005), and immunomodulators (Dancey, J. E. et al., Clin Adv Hematol Oncol 1:419-423, 2003) have intracellular site of action. There are a number of biological barriers to cellular drug delivery (Panyam, J. and Labhasetwar, V., Adv Drug Deliv Rev 55:329-347, 2003; Panyam, J. and Labhasetwar, V., Curr Drug Deliv 1:235-247, 2004). Simple diffusion across the cell membrane is feasible for only low molecular weight lipophilic drugs. Most drug molecules, however, are weak acids or bases, containing at least one site that may reversibly disassociate or associate a proton to form a negatively charged anion or a positively charged cation at physiologic pH (Martin, A. et al., Physical pharmacy. Physical chemical principles in the pharmaceutical sciences, Waverly International, Baltimore, 1993). Because the cell membrane is lipophilic and limits the diffusion of compounds that are ionized or polar, availability of many drugs at their intracellular site of action is limited. For drug molecules that get into the cell, cellular concentrations are maintained only as long as the concentration (or activity) gradient is maintained outside the cells. Once the concentration gradient is removed, drugs diffuse back out of the cell rapidly (Panyam, J. and Labhasetwar V., Mol Pharm 1:77-84, 2004; Suh, H. et al., J Biomed Mater Res 42:331-338, 1998). As a result, a single-dose administration of most drugs results in only a transient therapeutic effect (Panyam, J. and Labhasetwar V., Mol Pharm 1:77-84, 2004).

Based on the fact that many drugs and compounds have intracellular sites of action, there is a significant need in the art for compositions and methods to ensure the sustained availability of compounds to cells and tissues.

### SUMMARY OF THE INVENTION

The invention disclosed herein relates to compositions and methods utilizing nanoparticles to facilitate sustained delivery of compounds into cells and tissues. Certain embodiments of the invention relate to nanoparticles comprising an anionic surfactant, such as aerosol OT (AOT) and a polysaccharide polymer alginate. Further embodiments relate to the use of nanoparticles to encapsulate water soluble drugs, such as doxorubicin, verapamil, diclofenac, and clonidine.

### BRIEF DESCRIPTION OF THE FIGURES

FIG. 1A shows the structure of alginate. The alginates shown are linear unbranched polymers containing β-(1→4)-linked D-mannuronic acid (M) and α-(1→4)-linked L-guluronic acid (G) residues. Alginates are not random copolymers but, according to the source algae, consist of blocks of similar and strictly alternating residues (i.e. MMMMMM, GGGGGG and GMGMGMGM).

FIG. 1B shows crosslinking and ‘egg-box’ formation of alginate in the presence of calcium salts.

FIG. 1C shows the structure of AOT with the sulfosuccinate head group and hydrocarbon tail group.

FIG. 1D shows the proposed structure of AOT-alginate nanoparticles. Inner core consists of alginate and AOT head groups crosslinked with calcium. This is surrounded by hydrocarbon tail groups of AOT. Gray squares represent drug molecules.

FIG. 2 shows the effect of concentration on surface tension of PVA solutions. The surface tension was measured using a KSV 2001 drop tensiometer. The surface tension values are an average of three values taken after digitizing each new droplet for 20 mins.

FIG. 3 shows the biphasic degradation of doxorubicin in phosphate buffered saline (PBS) at 37° C. and 100 rpm. The r² values for the two phases were 0.9890 (1-10 days) and 0.9926 (12-28 days).

FIG. 4 shows the in vitro release of doxorubicin, verapamil and clonidine in PBS at 37° C. and 100 rpm.

FIG. 5 shows simultaneous in vitro release of doxorubicin and verapamil in PBS at 37° C. and 100 rpm from nanoparticles loaded with both the drugs.

FIG. 6 shows the effect of salt concentration of release medium on in vitro release of verapamil. The release was conducted at 37° C. and 100 rpm.

FIG. 7 shows the in vitro release of diclofenac sodium in PBS at 37° C. and 100 rpm.

FIG. 8 shows the swelling kinetics of AOT-alginate nanoparticles. AOT and PVA concentrations were 20% w/v and 2% w/v, respectively.

FIG. 9 shows an in vitro release of doxorubicin from nanoparticles. Nanoparticles were dispersed in PBS (pH 7.4) and incubated in a shaker at 37° C. and 100 rpm. Drug concentrations in the release buffer was measured by HPLC. The release shown is from 300 μg of nanoparticles. Data are means±SD (n=3).

FIG. 10 shows cellular uptake of rhodamine 123. MDA-kb2 cells were incubated with rhodamine encapsulated in nanoparticles or in solution for 2 hrs at 37° C. in the presence of serum-containing medium. Cellular drug content was measured at different time intervals and was normalized to the total cell protein. Drug uptake was significantly higher (P<0.05, t-test, n=4) in cells treated with nanoparticles than with drug in solution for both the doses.

FIG. 11A shows the kinetics of nanoparticle uptake into cells. MDA-kb2 cells were incubated with various doses of rhodamine encapsulated in nanoparticles for 2 hrs at 37° C. Cellular drug content was measured and was normalized to the total cell protein.

FIG. 11B shows the kinetics of nanoparticle uptake into cells. Cells were incubated with 100 μg/mL of nanoparticles for different time intervals at 37° C. Cellular drug content was measured and was normalized to the total cell protein.

FIG. 12 shows a mechanism of nanoparticle uptake into cells. MDA-kb2 cells were incubated with rhodamine encapsulated in nanoparticles for 2 hrs in the presence or absence of metabolic inhibitors 0.1% w/v sodium azide and 50 mM 6-deoxyglucose at 37° C. or 4° C. in serum-containing medium. Cellular drug content was measured and was normalized to the total cell protein. Drug uptake was significantly lower (P<0.05, t-test, n=4) in cells treated with metabolic inhibitors and at lower temperature.

FIG. 13 shows the cellular retention of rhodamine 123. MDA-kb2 cells were incubated with rhodamine in nanoparticles or in solution for 2 hrs. At the end of 2 hrs, cells were washed to remove uninternalized drug and added with fresh medium. Cellular drug content was measured at different time intervals and was normalized to the total cell protein. Data are represented as a percent of R123 levels at the end of 2-hr incubation. Cells treated with nanoparticles demonstrated higher drug retention than cells treated with drug in solution. (*P<0.05, t-test, n=4)

FIG. 14 shows enhanced cytotoxicity with doxorubicin nanoparticles. MCF-7 cells were plated in 96-well plates at 5,000 cells/well/0.1 ml. On Day 0, cells were treated with doxorubicin in solution or encapsulated in nanoparticles. Untreated cells and blank nanoparticle-treated cells were used as controls. On Day 2, cells were washed to remove the treatments and added with fresh medium with no further dose of treatments added. Cytotoxicity was followed using a MTS assay (Promega). Cell proliferation presented as a percent of respective controls (n=6).

FIG. 15. Nanoparticles enhanced tumor accumulation of encapsulated drug. Tumors were initiated in female Balb/c mice by subcutaneous injection of JC cell suspension (10⁶ cells in 0.1 ml PBS). Mice that developed tumors of at least 100 mm³ volume were injected intravenously with treatments equivalent to 4 mg/kg dose of rhodamine 123 (R123). Mice were euthanized at the end of six and seventy two hours, and tumors were excised. Tissues were homogenized, lyophilized, and extracted with methanol. Rhodamine concentration was analyzed by HPLC and was normalized to dry weight of the organ. (*P<0.05; n=4-5)

FIG. 16. Nanoparticle-mediated combination PDT and chemotherapy overcame tumor drug resistance in vivo. Female Balb/c mice bearing JC tumors of at least 100 mm³ volume were injected intravenously with treatments equivalent to 8 mg/kg dose of methylene blue and 4 mg.kg doxorubicin. About twenty four hours after treatment administration, tumors were exposed to light of 665 nm wavelength (50 J/cm²). Animals were then monitored for tumor growth. The results are shown as percent increase in tumor volume as a function of time after treatment (days), with the various treatment protocols.

FIG. 17. Nanoparticle-mediated combination therapy induced both necrosis (Top Row) and immune response (Bottom Row). Female Balb/c mice bearing JC tumors were injected intravenously with treatments equivalent to 8 mg/kg dose of methylene blue and 4 mg/kg doxorubicin and exposed to light (665 nm wavelength; 50 J/cm²). Animals were euthanized, and the excised tumor samples were processed for H&E (Top Row) or TUNEL (Bottom Row). Paired samples are shown in 100 and 400-fold magnification. FIGS. 17A and B, and FIGS. 17E and F, Dox NP; FIGS. 17C and D, and FIGS. 17G and H, Dox/MB NP. Nec=necrosis, Apo=apoptosis.

FIG. 18. Nanoparticle-mediated combination therapy reduced tumor cell proliferation (Top Row) and angiogenesis (Bottom Row). Female Balb/c mice bearing JC tumors were injected intravenously with treatments equivalent to 8 mg/kg dose of methylene blue and 4 mg/kg doxorubicin and exposed to light (665 nm; 50 J/cm²). Animals were euthanized, and the excised tumor samples were processed for PCNA expression (Top Row) or CD34 (Bottom Row) staining. Dox NP: FIGS. 8A and B, 200 and 400 times magnification; FIGS. 18E and F, 100 and 400 times magnification. Dox/MB NP: FIGS. 18C and D, 200 and 400 times magnification; FIGS. 18G and H, 100 and 400 times magnification.

FIG. 19. Enhanced cytotoxicity with doxorubicin nanoparticles in (A) MCF-7 cells and (B) NCI-ADR/RES cells. Cells were treated with blank nanoparticles (BLANK NP), doxorubicin in solution (DOX Soln), or doxorubicin in nanoparticles (DOX NP). Results are expressed as means (the standard error from three independent experiments, each performed in duplicate).

FIG. 20. Sustained cytotoxicity with doxorubicin nanoparticles in NCI-ADR/RES cells. Cells were incubated with doxorubicin in solution (0.4 μg/mL), doxorubicin and verapamil (23.0 μg/mL) in solution (DOX+Ver Solution), doxorubicin in nanoparticles (equivalent to 0.4 μg/mL doxorubicin), or doxorubicin and verapamil in nanoparticles (DOX+Ver NP; equivalent to 0.4 μg/mL doxorubicin and 23.0 μg/mL verapamil). An asterisk indicates a P of <0.05 vs untreated cells (n) 6).

FIG. 21. Cellular accumulation of rhodamine 123 (R123) in NCI-ADR/RES cells (n) 4). The asterisk indicates a P of <0.05 (t test).

FIG. 22. Effect of nanoparticle dose on rhodamine 123 (R123) accumulation in (A) MCF-7 cells and (B) NCIADR/RES cells. Cells were incubated with various doses of nanoparticles containing rhodamine for 2 h (n) 4). (C) Energy dependence of nanoparticle uptake in NCI-ADR/RES cells. Data are means (the standard deviation (n) 4). An asterisk indicates a P of <0.05 compared to control (nanoparticle treatment at 37° C. and in the absence of inhibitors) (t test).

FIG. 23. Intracellular distribution of doxorubicin. NCI-ADR/RES cells were treated with blank nanoparticles (A), doxorubicin in solution (B and D), or doxorubicin in nanoparticles (C and E) for 2 h. Cells were rinsed, counterstained with DAPI, and imaged by fluorescence microscopy (A-C). The magnification is 40×. In panels D and E, cells were also incubated with 75 nM Lysotracker Green for 30 min at 37° C. before being imaged. The magnification is 100×. Free doxorubicin is present near the cell surface (arrow in panel D) and is localized in endocytic vesicles. In the case of nanoparticles, a majority of doxorubicin is endocytosed and is present inside the cells rather than at the cell surface, extending all the way to the nucleus (arrows in panels C and E).

FIG. 24. Effect of blank nanoparticles on the accumulation of (A) rhodamine 123 (R123) and (B) fluorescein in NCI-ADR/RES cells. Cells were incubated with a mixture of blank nanoparticles and rhodamine or fluorescein in solution for 2 h (n) 4). The asterisk indicates a P of <0.05 (t test).

### DETAILED DESCRIPTION OF THE INVENTION

Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described.

The invention disclosed herein relates to compositions and methods utilizing nanoparticles for the sustained delivery of drugs or therapeutic agents to cells, including cells of the skin. As described herein, nanoparticles comprise a copolymer, such as alginate, and a surfactant, such as aerosol OT (AOT), and may further comprise an encapsulated drug or therapeutic agent. Such nanoparticles may promote increased delivery of drugs to intracellular targets, as well as allow drug delivery to occur in a sustained-release manner. Because of sustained release properties, nanoparticles may prolong the cellular availability of an encapsulated drug, resulting in greater and sustained therapeutic effect. These nanoparticles may positively affect human health by leading to improved treatment outcomes for diseases such as cancer and psoriasis, and for wound care, including traumatic wounds and surgical wounds. Non-limiting examples of such dermal conditions and wounds are disclosed in U.S. Pat. No. 6,025,150, which is incorporated herein in its entirety. The inventive nanoparticles are novel because (1) sustained, zero-order release of water-soluble drugs from nanocarriers has not been demonstrated before, (2) the use of electrostatic interactions is a novel approach to control drug release, and (3) currently there is no delivery system available to sustain the cellular delivery of water-soluble drugs in both drug-sensitive and resistant cells.

Since nanoparticles are often polymeric in nature and generally submicron in size, they have advantages in drug delivery. Nanoparticles may be used to provide targeted (cellular/tissue) delivery of drugs, to improve oral bioavailability, to sustain the effects of drugs or therapeutically-administered genes on target tissue, to solubilize drugs for intravascular delivery, and to improve the stability of therapeutic agents against enzymatic degradation (nucleases and proteases), especially of protein, peptide, and nucleic acid drugs. The nanometer size-range of these delivery systems offers advantages for drug delivery. Due to their sub-cellular and sub-micron size, nanoparticles may penetrate deep into tissues and are generally taken up efficiently by cells. This allows efficient delivery of therapeutic agents to target sites in the body. Nanoparticles may penetrate into small capillaries, allowing enhanced accumulation of the encapsulated drug at target sites (Calvo, P. et al., Pharm. Res. 18:1157-1166, 2001). Nanoparticles may also passively target tumor tissue through enhanced permeation and retention effect (Monsky, W. L. et al., Cancer. Res. 59:4129-4135, 1999; Stroh, M. et al., Nat. Med. 11:678-682, 2005). Also, by modulating polymer characteristics, it is possible to control the release of a therapeutic agent from nanoparticles to achieve desired therapeutic level in target tissue for the required saturation for optimal therapeutic efficacy. Further, nanoparticles may be delivered to distant target sites by localized delivery using a minimally-invasive catheter-based approach (Panyam, J. et al., Faseb J. 16:1217-1226, 2002).

The inventors have developed a novel nanoparticle formulation for the encapsulation of water-soluble drugs with high efficiencies, up to 100%. In addition, these nanoparticles demonstrate sustained release of water-soluble drugs over a period of weeks (˜60-80% of encapsulated drug released over a period of 4 weeks). Further, by changing the various formulation parameters it is possible to modulate drug loading and the rate and extent of drug release from nanoparticles. This will enhance the therapeutic efficacy of drugs that have intracellular sites of action.

As used herein, the term “nanoparticle” (also known as nanosphere) refers to sub-micron sized particles comprising a dense polymeric network. Nanoparticles useful for the applications disclosed herein are generally in the 10-1000 nanometer size range, for example the 30 to 500 nanometer size range, and the 50-350 nanometer size range. These ranges are exemplary only and not limiting for any particular application or route of administration, including intranasal, bucal, suppository, dermal, oral, and intravenous. The polymeric network may be used to encapsulate a drug or therapeutic agent. Also included are nanocapsules, which are formed by a thin polymeric envelope surrounding a drug-filled cavity (Garcia-Garcia, E. et al., Int J Pharm 298:274-292, 2005).

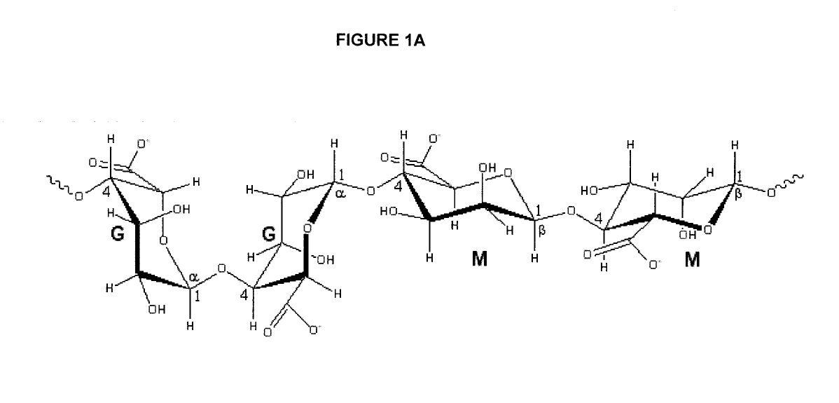

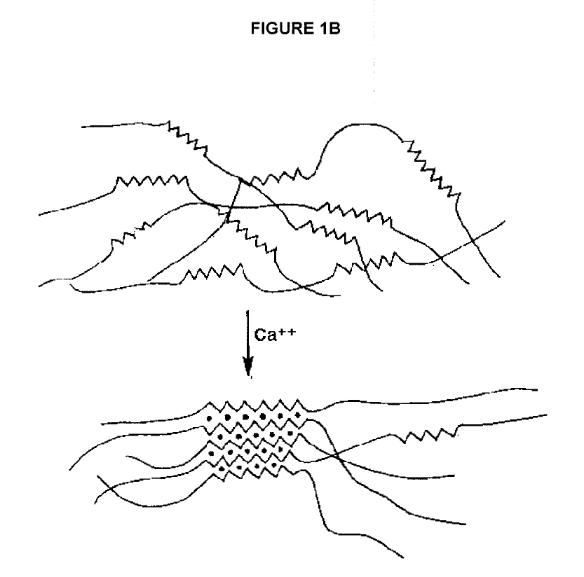

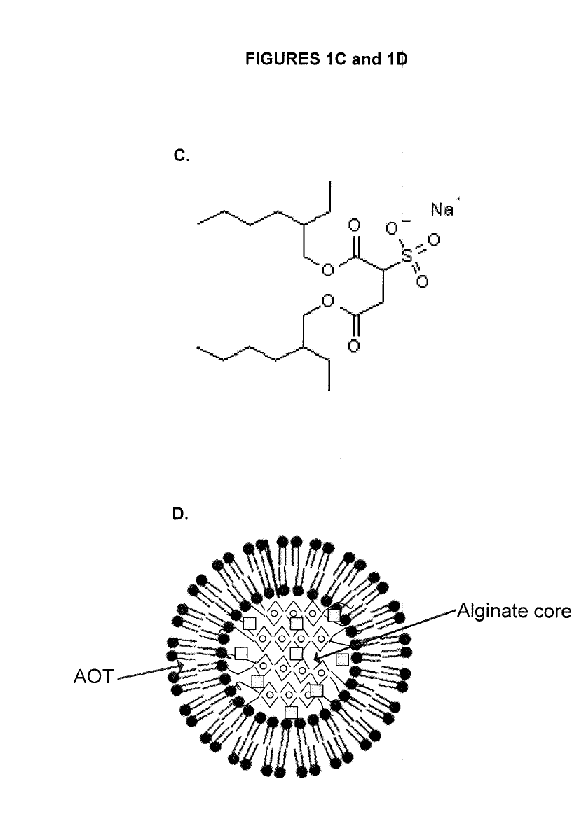

Particular embodiments of the invention relate to the use of AOT-alginate nanoparticles. Alginates are naturally occurring, random, anionic, linear polymers consisting of varying ratios of guluronic and mannuronic acid units (FIG. 1A). Alginate delivery systems are formed when monovalent, water-soluble, salts of guluronic and mannuronic acid residues undergo aqueous sol-gel transformation to water-insoluble salts (FIG. 1B) due to the addition of divalent ions such as calcium (Gombotz et al, 1998). Calcium ions have a greater affinity for guluronic acid than for mannuronic acid units (Gombotz, W. R. and Yee, S., Adv Drug Deliv Rev 31:267-285, 1998). As a result, calcium ions initially react with repeating guluronic acid units to form an ‘egg-box’ structure (FIG. 1B) that generally stack upon each other (Gombotz, W. R. and Yee, S., Adv Drug Deliv Rev 31:267-285, 1998; Skaugrud, O. et al., Biotechnol Bioeng 16:23-40, 1999). A limitation of prior alginate-based nanoparticles is that they lead to rapid drug release in physiologic salt concentration (De, S. and Robinson, D. H., J Control Release 89:101-112, 2003). In the presence of monovalent (e.g., sodium) salts, insoluble calcium alginate rapidly converts into soluble sodium alginate, resulting in immediate disintegration of the delivery system and drug release (De, S. and Robinson, D. H., J Control Release 89:101-112, 2003).

The inventive nanoparticles ameliorate this issue by incorporating stronger acid groups in the nanoparticle matrix, resulting in stronger cross-linking, slower degradation of the matrix, and stronger drug-matrix interaction. Based on this rationale, a hybrid surfactant-polymer system composed of alginate and anionic surfactant AOT (docusate sodium) has been engineered and disclosed herein. AOT has a sulfonic group (pKa<1) in its polar sulfosuccinate head group with a large and branching hydrocarbon tail group (FIG. 1C). AOT forms reverse micelles in non-polar solvents. Based on these properties, a multiple emulsion-crosslinking technology to form AOT-alginate nanoparticles has been designed.

To produce nanoparticles with the desired properties, an aqueous solution of drug may be emulsified with sodium alginate in a chloroform solution of AOT. This simple emulsion is then further emulsified into an aqueous polyvinyl alcohol solution, resulting in a multiple water-in-oil-in-water emulsion. Because AOT is a double chain amphiphile, it is expected to form a bilayered structure in the multiple emulsion (Israelachevilli, J., Intermolecular and Surface Forces, 2nd edn, London, Academic Press, 1991). The multiple emulsion may then be crosslinked with calcium chloride. The chloroform may be evaporated, resulting in the formation of AOT-alginate nanoparticles. The nanoparticles have a calcium-crosslinked core composed of alginate and AOT head groups, surrounded by a hydrophobic matrix composed of AOT tails, with the drug of interest encapsulated in the core (FIG. 1D). The term “drug of interest” is not limiting, and includes water-soluble drugs such as cancer drugs, antibiotics, and polypeptidic compounds including proteins, polypeptides, and antibodies.

Contact angle measurements may be taken to demonstrate that the surface of nanoparticles are hydrophilic, indicating the presence of polar head groups of AOT on the surface (FIG. 1D). AOT has been shown to be easily removed from the body through renal elimination, and does not accumulate even after multiple dosing (Kelly, R. G. et al., The pharmacokinetics and metabolism of dioctyl sodium sulfo-succinate in several animal species and man: report submitted to WHO [Lederle Laboratories, American Cyanamid, 1973]).

Nanoparticles, such as AOT-alginate nanoparticles, may be used to encapsulate a wide array of drugs or therapeutic agents. The inventive nanoparticles particularly allow for the encapsulation of hydrophilic and water-soluble drugs. For example, nanoparticles may be used to encapsulate doxorubicin, verapamil, clonidine, diclofenac, and rhodamine, as well as compounds comprising peptide, proteins, nucleic acids, or combinations thereof. Any number of other compounds may be utilized with the inventive nanoparticles, as will be appreciated by those of skill in the art. Encapsulation of other drugs or therapeutic agents may be achieved by a skilled artisan using procedures outlined herein without undue experimentation.

Skin diseases and conditions are amenable to treatment using methods and compositions as disclosed herein, for example, topical compositions for the treatment of psoriasis or other skin disorders such as dry skin, eczema, itchy skin, red skin, itchy eczema, inflamed skin, and/or cracked skin. Psoriasis is characterized generally by the presence of skin elevations and scales which may be silvery in appearance. Psoriasis can accelerate the epidermal proliferation and proliferation of capillaries in the dermal region. In addition, psoriasis frequently results in the evasion of the dermis and epidermis by inflammation of the affected cells. Thus, psoriasis is suitable for treatment using nanoparticles described herein which provide sustained release of one or more drugs or compounds effective against the psoriatic lesions. Examples of such drugs and compounds include Anthralin, Dovonex, Taclonex, Tazorac, topical steroids, and salicylic acid. Drugs may be administered orally, intravenously, transdermally, via mucosal route, or via nasal spray. However, the list is non-limiting, and nanoparticle delivery is useful for other compounds and drugs in treating psoriasis and skin conditions and diseases.

Other suitable drugs include Domperidone and fluticasone propionate for gastrointestinal treatments by oral route of administration. Methotrexate, cyclosporine, and other steroids are suitable for treating psoriasis as topical therapy. Polypeptide compounds are also suitable. A non-limiting example is the peptide PHSRN (Pro His Ser Arg Asn) and derivatives thereof, which are disclosed in U.S. Pat. No. 6,025,150, incorporated herein by reference. One example is peptide Ac—PHSRN—NH₂.

Within the context of the nanoparticle formulations herein, it is not intended that the present invention be limited by the particular nature of the therapeutic preparation, so long as the preparation comprises at least one suitable therapeutic agent or drug, with or without an imaging agent as appropriate. For example, such compositions can be provided together with physiologically tolerable liquid, gel or solid carriers, diluents, adjuvants and excipients. These nanoparticle preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual animal or patient. Such dosages are within the skill of the practitioner or clinician.

Drug-encapsulated nanoparticle compositions may be introduced into a recipient by any suitable means. For example, such compositions may be administered intravenously, intraperitoneally, or via a catheter-type system. Such compositions may be used for any medical condition requiring intracellular drug delivery. Another application of drug-encapsulated nanoparticles involves the use of photodynamic therapy (PDT). PDT in solid tumors for detection and treatment has been investigated since the early twentieth century. (Wiedmann, M. W. and Caca, K., Current pharmaceutical biotechnology 2004; 5:397-408; Ackroyd, R. et al., Photochemistry and photobiology 2001; 74:656-69.) Currently, PDT is used in the clinic as an adjunctive treatment in a variety of solid tumors including inoperable esophageal tumors, head and neck cancers, and microinvasive endo-bronchial non-small cell lung carcinoma. (Brown, S. B. et al., The Lancet Oncology 2004; 5:497-508.) In addition, PDT is being considered as an alternative and promising approach for the treatment of breast cancer. (Dolmans, D. E. et al., Photodynamic therapy for cancer, Nature reviews 2003; 3:380-7; Allison, R. et al., Cancer 2001; 91:1-8.) PDT has shown promising preliminary clinical results in the treatment of breast cancer and in the treatment of cutaneous and subcutaneous breast cancer metastases. The use of PDT is based on the fact that certain compounds, called photosensitizers (PS), selectively accumulate in solid tumors and can induce cell death following activation by light. (Diamond, I. et al., Lancet 1972; 2:1175-7.)

In the presence of molecular oxygen, exposure of a photosensitizer to light of a specific wavelength, which is around its absorption spectrum, activates that compound. You, Y. et al., Journal of medicinal chemistry 2003; 46:3734-47; An, H. et al. Free radical research 2003; 37:1107-12; Chekulayeva, L. V. et al., J Environ Pathol Toxicol Oncol 2006; 25:51-77. Activated photosensitizer generates singlet oxygen species (¹O₂) and other reactive oxygen species (ROS). ROS generation is the main mechanism of cytotoxicity in PDT. Combination of different cytotoxic events are responsible for PDT-mediated tumor destruction; direct cell kill caused by oxidative DNA damage and single DNA strand breakage (Viola, G. et al., Chemical research in toxicology 2003; 16:644-51), damage to the tumor's vasculature (Krammer, B., Anticancer research 2001; 21:4271-7; Heckenkamp, J. et al., Arteriosclerosis, thrombosis, and vascular biology 1999; 19:2154-61) and induction of an immune response (Krosl, G. et al., British journal of cancer 1995; 71:549-55).

Methylene blue is a water-soluble phenothiazine derivative PS that efficiently generates singlet oxygen species and other ROS and induces cell death. (Trindade, G. S. et al., Cancer Lett 2000; 151:161-7; Capella, M. A. and Capella, L. S., J Biomed Sci 2003; 10:361-6; Roy, I. et al., J Am Chem Soc 2003; 125:7860-5) Methylene blue has a variety of applications; it is used as an oxidation-reduction indicator (Miclescu, A. et al., Critical care medicine 2006; 34:2806-13; Furian, A. F. et al., Neurochemistry international 2007; 50:164-71) an antidote in cyanide toxicity (Aly, F. W., Arztliche Wochenschrift 1957; 12:1014-8), as a diagnostic dye in certain conditions such as localization of lymph nodes (Varghese. P. et al., Eur J Surg Oncol 2006), and as a disinfectant (Wainwright, M., International journal of antimicrobial agents 2000; 16:381-94). Clinical use of MB in PDT, after resection procedure in patients with local esophageal tumors, showed successful recession of tumors with no clinical complications. Orth, K. et al., Lancet 1995; 345:519-20. MB is approved by Food and Drug Administration (FDA) for clinical intravenous administration in treatment of methemoglobinemia. Wendel, W. B., J Clin Invest 1939; 18:179-85. In addition, recent studies have shown that methylene blue may also be able to modulate P-glycoprotein (P-gp), a major efflux transporter, and overcome tumor resistance to chemotherapeutic drugs that are P-gp substrates. Trindade, G. S. et al., Cancer Lett. 151:161-167, 2000.

Clinical use of methylene blue for PDT has been limited because of the lack of activity following systemic injection. This is due in part to its relatively poor accumulation into the tumor cells. In addition, once in the biological environment, methylene blue is extensively up-taken by erythrocytes (Sass, M. D. et al. The Journal of laboratory and clinical medicine 1967; 69:447-55) and endothelial cells (Bongard, R. D. et al., The American journal of physiology 1995; 269:L78-84; Olson, L. E. et al., Annals of biomedical engineering 2000; 28:85-93) where it is inactivated by reduction to neutral leucomethylene blue, which has negligible photodynamic activity (Gabrielli, D. et al., Photochemistry and photobiology 2004; 79:227-32). One approach to overcome these limitations is to encapsulate methylene blue in drug delivery systems such as nanoparticles (Tang, W. et al., Photochemistry and photobiology 2005; 81:242-9) or liposomes (Takeuchi, Y. et al., Bioconjugate chemistry 2003; 14:790-6). Results described in Example 4 below indicate that that AOT-alginate nanoparticles enhance methylene blue-mediated PDT in model tumor cell lines in vitro.

Currently, PDT is known as an efficient treatment modality for cancer and psoriasis. World wide, PDT is clinically approved as an adjunctive treatment in a variety of solid tumors, especially in conditions were other treatment modalities have failed or are inappropriate. This includes inoperable esophageal tumors, head and neck cancers, skin tumors and microinvasive endo-bronchial non-small cell lung carcinoma. PDT can be described as a photo-toxicity process utilizing three elements at the same time; light, oxygen and chemical compounds called photosensitizers (PS). Photosensitizers selectively accumulate in solid tumors and induce cell death following activation by light. In the presence of molecular oxygen, exposure of a photosensitizer to light of a specific wavelength, that is around its absorption spectrum, results in the compound's absorption of light and conversion into an excited state. Chekulayeva, L. V. et al., J Environ Pathol Toxicol Oncol 2006; 25:51-77.

An excited state is a high-energy, long-lived triplet state a photosensitizer acquires upon absorption of photons in the ground state. In most cases at the triplet state level of energy, a photosensitizer is considered to be an activated photosensitizer. Generation of ROS by activated photosensitizers is the main mechanism of cytotoxicity in PDT. Two different pathways for ROS formation have been reported in PDT. When an excited PS returns to the ground state, it transfers energy to molecular oxygen causing the formation of singlet oxygen species (Type-II reaction). Excited photosensitizer may also transfer electrons to existing compounds other than oxygen such as lipid membrane components, nitric oxide and hydroxyl groups forming free radicals and radical ions of these compounds which can then interact with molecular oxygen to form oxygenated products (Type-I reaction). PDT is a potent method to induce apoptosis in susceptible cells (Kessel, D. and Luo, Y., Cell death and differentiation 1999; 6:28-35) as well as active death in cells that lost the ability to undergo apoptosis especially after radio- or chemotherapy (Stewart, F. et al., Radiother Oncol 1998; 48:233-48). In addition, it has been reported that PDT can damage the tumor microvessels which reduces tumor's blood supply. Krammer, B., Anticancer research 2001; 21:4271-7; Heckenkamp, J. et al., Arteriosclerosis, thrombosis, and vascular biology 1999; 19:2154-61). Others have reported induction of the immune system after PDT. (Krosl, G. et al., British journal of cancer 1995; 71:549-55).

Methylene blue (MB) is a positively charged, water-soluble phenothiazine derivative PS that efficiently generates singlet oxygen species and other ROS upon activation with light of wavelength around 668 nm. Activated methylene blue has been shown to deliver (¹O₂) directly inside tumor cells leading to oxidative DNA damage, single-strand DNA breaks, and cell death through induction of apoptosis. As a photosensitizer, MB was successfully used in clinic for local treatment of inoperable esophageal tumors. However, the use of MB in PDT has been largely limited by the lack of activity following systemic injection. This has resulted from the poor accumulation of active (oxidized) MB in tumor cells. Poor tumor availability can be partially explained by the extensive uptake of MB by the erythrocytes and endothelial cells. In these cells, following systemic administration, thiazine dyes are extensively reduced to, for example MB+ is reduced to (MBH) leucomethylene blue. A specific enzymatic system called thiazine dye reductase has been described to mediate cellular reduction and uptake of MB. Once it is reduced MB looses its inherent photo-sensitizing activity. In addition, it has been reported that (MB+) is also reduced by extracellular reductants. Therefore, there is a crucial need for the delivery of MB in its oxidized integrity to tumor cells as well as limiting its inactivation by thiazine dye reductase and uptake by erythrocytes and endothelial cells.

The in vitro studies described below showed that photo-activated methylene blue loaded in nanoparticles was significantly more effective than that in solution. In addition, the in vitro cytotoxic effect increased with increased dose of MB and/or light. For example, cytotoxicity with nanoparticle-encapsulated 0.6 μM MB was more significant than that with 0.3 μM. In addition, MB loaded in nanoparticles was significantly more effective than MB in solution at equivalent doses. On the other hand, induced cell death with MB-loaded nanoparticles was significantly higher at 2400 mJ/cm² dose of light than at 1200 mJ/cm². This indicated a dose-response cytotoxic effect of light. The use of nanoparticles as a drug carrier provides protection for encapsulated drug(s) from harsh environments such as enzymatic metabolism. (Damge, C. et al., Journal of pharmaceutical sciences 1997; 86:1403-9; He, X. X. et al., Journal of the American Chemical Society 2003; 125:7168-9) Nanoparticles also increase drug accumulation in solid tumors through the enhanced permeation and retention effect. Iyer, A. K. et al., Drug discovery today 2006; 11:812-8. It has been reported that nanoparticles in the sub-micron size are endocytosed into tumor cells which enhances intracellular accumulation of the nanoparticle-encapsulated drug. Brannon-Peppas, L. et al., Adv Drug Deliv Rev 2004; 56:1649-59; Yoo, H. S. et al., J Control Release 2000; 68:419-31. According to the present disclosure, MB-loaded AOT-alginate nanoparticles were fabricated with an average size around 72 nm in diameter and a net negative surface charge of around −20 mV. Nanoparticles with a negative surface charge have the advantage of stability in buffer and medium containing serum. Tiyaboonchai, W. and Limpeanchob, N., International journal of pharmaceutics 2007; 329:142-9; Howe, A. M. et al., Langmuir 2006; 22:4518-25.

The present cellular accumulation studies showed that the use of AOT-alginate nanoparticles resulted in significantly higher intracellular levels of methylene blue than that in solution. This indicates that enhanced cellular accumulation of methylene blue resulted in enhanced PDT. Previous studies have demonstrated that diffusion of ionized compounds through the cell membrane is highly restricted limiting the availability of ionized drugs at their intracellular site of action. Methylene blue has a basic pKa which renders a strong positive charge in vivo. Ziv, G. and Heavner, J. E., Journal of veterinary pharmacology and therapeutics 1984; 7:55-9.

According to the present disclosure, encapsulation of MB in nanoparticles resulted in enhanced production of ROS. It also resulted in increase production of singlet oxygen species. Ex vitro ROS studies showed that photo-activated MB loaded in nanoparticles generated significantly higher ROS yields than that in solution. For example, nanoparticle-encapsulated MB produced around 2-fold higher ROS yield than MB solution at two different doses of the drug (0.3 μM and 0.6 μM). Previous studies have reported that generation of ROS is the main mechanism of cytotoxicity in PDT. Lu, Z. et al., Free radical biology & medicine 2006; 41:1590-605; Diwu, Z. and Lown, J. W., Journal of photochemistry and photobiology 1993; 18:131-43. However, Weishaupt et al. reported that (¹O₂) are the main cytotoxic species in PDT. Weishaupt, K. R. et al., Cancer research 1976; 36:2326-9.

Other studies have reported that ROS yield in target cells depends on the cellular level of PS (Sheng, C. et al., Photochemistry and photobiology 2004; 79:520-5), dose of light (McCaughan, J. S. Jr. et al., The Annals of thoracic surgery 1992; 54:705-11; Fingar, V. H. and Henderson, B. W., Photochemistry and photobiology 1987; 46:837-41), and cellular level of molecular oxygen (Vaupel, P. and Harrison, L., The oncologist 2004; 9 Suppl 5:4-9; Johansson, A. J. et al., Journal of biomedical optics 2006; 11:34029). On the other hand, Vakrat-Haglili et al. reported that the microenvironment surrounding the PS during light illumination significantly affect ROS generation both in vitro and in vivo. Vakrat-Haglili, Y. et al., Journal of the American Chemical Society 2005; 127:6487-97. This included molecular oxygen (Alvarez, M. G. et al., The international journal of biochemistry & cell biology 2006; 38:2092-101), and other surrounding compounds (Chekulayeva, L. V. et al., Free Radical Res. 37:1107-1112, 2003), pH 9 (Bronshtein, I. et al., Photochemistry and photobiology 2005; 81:446-51), and hydrophobicity of the milieu (Cao, Y. et al., Photochemistry and photobiology 2005; 81:1489-98; Rotta, J. C. et al., Brazilian journal of medical and biological research 2003; 36:587-94).

Furthermore, PS encapsulated or attached to drug carriers does not need to dissociate from its carriers for light-activation to occur. (Tang, W. et al., Photochemistry and photobiology 2005; 81:242-9). Nanoparticles used in the present Examples were composed of alginate and Aerosol-OT. Without being bound by a particular mechanism, both molecules possess many functional groups that might be candidate acceptors of electron(s) from activated MB for ROS generation. For example, alginates are polysaccharide polymers that consist of alternative sugar units of guluronic and mannuronic acids which have free carboxylic acid groups. These free carboxylic groups might accept electron(s) from activated methylene blue which results in generation of ROS. This might partially explain the higher yield of ROS generated with MB in nanoparticles compared to that with the free drug. The present results might be explained by the enhanced cellular uptake and retention with the use of nanoparticles. Also nanoparticles might provide a protection for MB from enzymatic degradation. In addition, nanoparticles might also provide an ideal microenvironment for ROS production.

Another use for the nanoparticles described herein is for overcoming multidrug resistance. Development of simultaneous resistance to multiple drugs, termed multidrug resistance (MDR), is a frequent phenomenon in cancer cells. (Stein, W. D. et al., Curr. Drug Targets 2004, 5:333-346) The significance of this problem is highlighted by the estimations that up to 500,000 new cases of cancer each year will eventually exhibit a drug-resistant phenotype. (Shabbits, J. A. et al., Expert Rev. Anticancer Ther. 2001, 1:585-594) Overexpression of drug transporters, stress response proteins, antiapoptotic factors, or other cellular proteins in tumor cells results in the development of MDR. Overexpression of P-glycoprotein (P-gp), a membrane-bound efflux pump and a product of the ABCB1 (MDR1) gene, is a key factor contributing to MDR. (Szakacs, G. et al., Nat. Rev. Drug Discovery 2006, 5:219-234) Expression of P-gp leads to energy-dependent drug efflux and a reduction in intracellular drug concentration. While the exact mechanism by which P-gp interacts with its substrate is not fully understood, it is thought that binding of a substrate to the high-affinity binding site results in ATP hydrolysis, causing a conformational change that shifts the substrate to a lower-affinity binding site and then releases it into the extracellular space. (Sauna, Z. E. et al., J. Bioenerg. Biomembr. 2001, 33:481-491)

Tumor cells that overexpress P-gp do not accumulate therapeutically effective concentrations of the drug and are, therefore, resistant to the drug's cytotoxicity. A number of studies demonstrate that P-gp-mediated drug efflux and MDR could be potentially overcome by the use of specific delivery systems.

As shown herein, AOT-alginate nanoparticles enhanced the cytotoxicity of doxorubicin significantly in drug-resistant cells. The enhancement in cytotoxicity with nanoparticles was sustained over a period of 10 days. Uptake studies with rhodamine-loaded nanoparticles indicated that nanoparticles significantly increased the level of drug accumulation in resistant cells at nanoparticle doses higher than 200 μg/mL. Blank nanoparticles also improved rhodamine accumulation in drug-resistant cells in a dose-dependent manner. Nanoparticle-mediated enhancement in rhodamine accumulation was not attributed to membrane permeabilization. Fluorescence microscopy studies demonstrated that nanoparticle-encapsulated doxorubicin was predominantly localized in the perinuclear vesicles and to a lesser extent in the nucleus, whereas free doxorubicin accumulated mainly in peripheral endocytic vesicles. As shown in Example 5 herein, an AOT-alginate nanoparticle system enhanced the cellular delivery and therapeutic efficacy of P-gp substrates in P-gp-overexpressing cells.

The Examples below are included for purposes of illustration only, and are not intended to limit the scope of the range of techniques and protocols in which the nanoparticles of the present invention may find utility, as will be appreciated by one of skill in the art and can be readily implemented.

### Examples

### Example 1

### AOT-Alginate Nanoparticles

AOT-alginate nanoparticles investigated in this study were developed for efficient encapsulation and sustained release of drugs or compounds, including water-soluble drugs like doxorubicin. In vitro release studies show that nanoparticles result in a near zero-order release of doxorubicin over a 15-day period. This Example shows that electrostatic interactions between weakly basic drug and anionic nanoparticle matrix composed of alginate and AOT contribute to the efficient encapsulation and sustained drug release properties of AOT-alginate nanoparticles. Following encapsulation of weakly basic drugs, nanoparticles have a net negative charge, which stabilizes nanoparticles in buffer and in medium containing serum. This is an advantage over other nanoparticle delivery systems such as polycyanoacrylate nanoparticles that become cationic following encapsulation of weakly basic drugs, such as doxorubicin.

### Materials and Methods

Materials: Doxorubicin, rhodamine 123, verapamil, methylene blue and clonidine (all hydrochloride salts), sodium alginate, polyvinyl alcohol (PVA, 30,000-70,000 Da) and calcium chloride were obtained from Sigma-Aldrich (St. Louis, Mo.). Fluorescein sodium, diclofenac sodium, AOT, ethanol and methylene chloride were obtained from Fisher Scientific (Chicago, Ill.). All salts and buffers were of reagent grade. Organic solvents were of HPLC grade.

### Methods

Nanoparticle formulation: Nanoparticles were formulated by emulsification-crosslinking technology. Sodium alginate solution in water (0.1% to 1.0% w/v; 1 ml) was emulsified into AOT solution in methylene chloride (0.05 to 20% w/v; 1 to 3 ml) by either vortexing (Genie™, Fisher Scientific) or sonication (Model 3000, Misonix, Farmingdale, N.Y.) for 1 min over ice bath. The primary emulsion was further emulsified into 15 ml of aqueous PVA solution (0.5 to 5% w/v) by sonication for 1 min over ice bath to form a secondary water-in-oil-in-water emulsion. The emulsion was stirred using a magnetic stirrer, and 5 ml of aqueous calcium chloride solution (60% w/v) was added slowly to the above emulsion. The emulsion was stirred further at room temperature for ˜18 hrs to evaporate methylene chloride. For preparing drug-loaded nanoparticles, drug (5 to 15 mg) was dissolved in the aqueous alginate solution, which was then processed as above. Nanoparticles formed were be recovered by ultracentrifugation (Beckman, Palo Alto, Calif.) at 145,000×g, washed two times with distilled water to remove excess PVA and unentrapped drug, resuspended in water, and lyophilized.

Determination of drug loading and encapsulation efficiency: Drug loading in nanoparticles was determined by extracting 5 mg of nanoparticles in 5 ml of 95% alcohol for 30 min and analyzing the alcohol extract for drug content. Methylene blue was quantified by spectrophotometry at 630 nm (Vmax, Molecular devices, CA); rhodamine and fluorescein were determined by fluorescence spectroscopy (excitation/emission wavelengths of 485/528 nm and 494/518 nm; FLX 8000, Bio-Tek® Instruments, Winooski, Vt.). All the other drugs were determined by HPLC (see below). Drug loading was defined as the amount of drug encapsulated in 100 mg of nanoparticles, and represented as % w/w. Drug encapsulation efficiency was calculated as a percent of the total drug added that was encapsulated in nanoparticles.

Determination of residual solvent content: According to USP29-NF24, methylene chloride is a Class 2 residual solvent, and its concentration in products is limited to 600 ppm. Residual methylene chloride content in selected nanoparticle formulations was determined by USP-NF OVI (Organic Volatile Impurities) Method IV Testing. The data was presented as ppm residual methylene chloride in nanoparticles.

Determination of particle size: Particle size of nanoparticles was determined by dynamic light scattering. About 1 mg of nanoparticles was dispersed in 1 ml of distilled water by sonication, and the particle size and zeta potential were determined in a particle size analyzer (90Plus, Brookhaven instruments, Holtsville, N.Y.). The particle size obtained is z-average particle size. Polydispersity index provides an estimate of particle size distribution.

In vitro release studies: In vitro release of nanoparticle-encapsulated drug was determined under sink conditions. The term “sink conditions” refers to release conditions in which the volume of the buffer used is sufficient to dissolve all of the drug present in the delivery system. Such conditions are used to assure that the amount of drug released is not limited by the degree of solubility in the buffer or solvent used. Nanoparticles (˜5 mg) were dispersed in 0.5 ml of phosphate-buffered saline (PBS, pH 7.4, 0.15M) and suspended in DispoDialyzer® (10 kDa MWCO, Pierce) dialysis tubes. These were then placed in a 15-ml centrifuge tube containing 10 ml of PBS. The whole assembly was shaken at 100 rpm and 37.0±0.5° C. in an orbital shaker (Brunswick Scientific, C24 incubator shaker, NJ). At predetermined time intervals, 0.5 mL of the dissolution medium was removed from the centrifuge tube, and was replaced with fresh buffer. Drug concentration in the release samples was determined as in drug loading determinations. Stability of different drugs under in vitro release conditions was determined and the drug release profile was corrected for degradation, if any.

HPLC analysis: A Beckman Coulter HPLC system with a binary pump system and an auto injector connected to PDA and fluorescence detectors were used for all the drugs. A Beckman® C-18 (Ultrasphere) column (ODS 4.6×250 MM) was used for all the drugs. The following mobile phase and detector wavelengths were used.

Doxorubicin:Acetonitrile:water (pH 3 adjusted with glacial acetic acid) at flow-rate of 1 ml/minute; and fluorescence detector at 505/550 nm wavelengths. Retention time—7 minutes.

Verapamil:Acetonitrile:sodium acetate (20 mM) pH 4: tetrabutylammonium bromide (1.5 mM) (50:20:30) at flow-rate of 1 ml/minute; and fluorescence detector at 275/310 nm wavelengths. Retention time—3.8 minutes.

Clonidine:Methanol:sodium 1-heptane-sulfonate (0.01 M) pH 3 (50:50) at a flow rate of 1 mL/min; and PDA detector at 220 nm. Retention time—8.0 min.

Diclofenac:Acetonitrile:sodium acetate (20 mM, pH 4): tetrabutylammonium bromide (1.5 mM) (6:1.6:2.4 ratio) at flow-rate of 1 mL/minute; and PDA detector at 280 nm. Retention time—6 minutes.

### Results.

Effect of formulation parameters on particle size: Particle size is often used to characterize nanoparticles, because it facilitates the understanding of the dispersion and aggregation processes. Further, particle size affects biologic handling of nanoparticles. For example, particles of size ˜100 nm have generally higher cellular uptake than that of ˜1 μm size particles (Desai, M. P. et al., Pharm. Res. 14:1568-1573, 1997). The effect of various formulation parameters on particle size of nanoparticles was studied.

In general, nanoparticles were in the size range of 200-300 nm. Changing sodium alginate or AOT concentration in the formulation did not significantly affect the particle size of nanoparticles as shown in Tables 1 and 2. However, increasing the PVA concentration in the emulsion from 0.5 to 5% w/v resulted in a decrease in the mean particle from 310 nm to 213 nm (Table 3). This decrease in particle size with increase in PVA concentration is probably due to the differences in the stability of emulsions formulated with different concentrations of PVA.

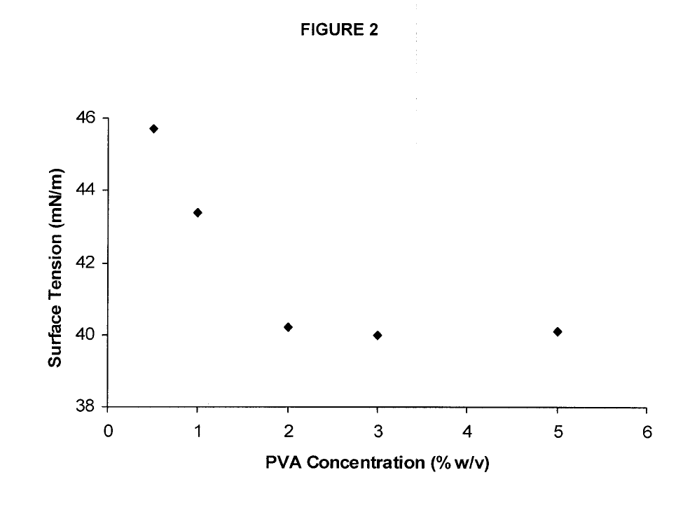

At concentrations less than 2.0% w/v, PVA exists as unimers in solution. Above this concentration, PVA forms aggregates (Tse, G. et al., J. Control. Release 60:77-100, 1999), with enhanced surface activity (FIG. 2). Further, the viscosity of PVA solution increases with increasing PVA concentrations (2.1 cps for 2% w/v to 5.7 cps for 5% w/v). Thus, increasing the PVA concentration in the formulation could have resulted in the formation of a more stable emulsion with smaller droplet size, resulting in the formation of smaller size nanoparticles (Sahoo, S. K. et al., J. Control. Release 82:105-114, 2002). A similar decrease in particle size with increase in PVA concentration has been observed for PLGA nanoparticles (Sahoo, S. K. et al., J. Control. Release 82:105-114, 2002).

TABLE 1
Effect of sodium alginate concentration on particle size,
zeta potential and methylene blue encapsulation ^{a}

| | Particle | Poly- | Drug | Encapsulation |
|---|---|---|---|---|
| Concentration | size | dispersity | loading | efficiency |
| (% w/v) | (nm) | Index | (% w/w) | (%) |
| 0.1 | 252.7 ± 2.2 | 0.205 | 0.63 ± 0.01 | 76.4 ± 0.8 |
| 0.3 | 230.9 ± 0.2 | 0.229 | 0.63 ± 0.01 | 76.2 ± 0.8 |
| 0.5 | 244.5 ± 3.4 | 0.276 | 0.65 ± 0.01 | 76.8 ± 0.2 |
| 0.7 | 219.8 ± 2.0 | 0.246 | 0.68 ± 0.01 | 83.0 ± 1.3 |
| 1.0 | 241.6 ± 4.3 | 0.262 | 0.82 ± 0.01 | 99.8 ± 0.6 |

^{a} AOT and PVA concentrations were 20% w/v and 2% w/v, respectively

TABLE 2
Effect of AOT concentration on particle size,
zeta potential and methylene blue encapsulation ^{a}

| | Particle | Poly- | Drug | Encapsulation |
|---|---|---|---|---|
| Concentration | size | dispersity | loading | efficiency |
| (% w/v) | (nm) | Index | (% w/w) | (%) |
| 0.05 | 224.5 ± 4.2 | 0.185 | 5.06 ± 0.24 | 16.7 ± 0.8 |
| 0.1 | 234.4 ± 6.2 | 0.236 | 4.44 ± 0.05 | 16.8 ± 0.2 |
| 5 | 228.2 ± 2.3 | 0.257 | 1.76 ± 0.02 | 58.2 ± 0.9 |
| 10 | 217.3 ± 3.9 | 0.197 | 1.38 ± 0.02 | 86.9 ± 1.2 |
| 20 | 241.6 ± 4.3 | 0.262 | 0.82 ± 0.01 | 99.8 ± 1.2 |

^{a} Sodium alginate and PVA concentrations were1% w/v and 2% w/v, respectively

TABLE 3
Effect of PVA concentration on particle size,
zeta potential and drug encapsulation ^{a}

| | Particle | Poly- | Drug | Encapsulation |
|---|---|---|---|---|
| Concentration | size | dispersity | loading | efficiency |
| (% w/v) | (nm) | Index | (% w/w) | (%) |
| 0.5 | 310.9 ± 2.2 | 0.220 | 0.71 ± 0.02 | 87.5 ± 2.7 |
| 1.0 | 324.9 ± 3.0 | 0.257 | 0.76 ± 0.02 | 93.4 ± 2.5 |
| 2.0 | 241.6 ± 4.3 | 0.262 | 0.82 ± 0.01 | 99.8 ± 0.6 |
| 3.0 | 255.9 ± 2.8 | 0.248 | 0.80 ± 0.02 | 98.8 ± 2.2 |
| 5.0 | 213.5 ± 1.3 | 0.265 | 0.80 ± 0.02 | 99.1 ± 2.0 |

^{a} AOT and sodium alginate concentrations were 20% w/v and 1% w/v, respectively

The effect of energy input on nanoparticle size was also investigated (Table 4). Increasing the energy during the first emulsification step did not significantly influence the particle size, because the primary emulsion step may affect only the size of the inner alginate droplets of the multiple emulsion and not the final emulsion droplet size. Increasing the sonication energy from 18 Watt to 48 Watt during the secondary emulsification step resulted in a decrease in the particle size from 241±4.3 nm to 192±3.9 nm (Table 4). Increasing the energy input during the secondary emulsification step probably resulted in a smaller droplet size of the secondary emulsion, resulting in a decrease in particle size. A similar decrease in particle size with increasing energy input has been observed for PLGA nanoparticles in previous studies (Panyam, J. et al., J. Control. Release 92:173-187, 2003).

TABLE 4
Effect of sonication energy on particle size,
zeta potential and drug encapsulation ^{a}

| Sonication energy | Particle | Poly- | Drug | Encapsulation |
|---|---|---|---|---|
| First/Second | size | dispersity | loading | efficiency |
| (Watt) ^{b} | (nm) | Index | (% w/w) | (%) |
| 0/18 | 241.6 ± 4.3 | 0.262 | 0.81 ± 0.01 | 99.8 ± 0.6 |
| 0/30 | 223.4 ± 4.4 | 0.236 | 0.67 ± 0.02 | 82.2 ± 2.6 |
| 0/48 | 192.8 ± 3.9 | 0.262 | 0.62 ± 0.01 | 75.5 ± 0.5 |
| 48/48 | 188.2 ± 3.7 | 0.225 | 0.54 ± 0.01 | 66.1 ± 1.6 |

^{a} Alginate, AOT and PVA concentrations were 1% w/v, 20% w/v and 2% w/v, respectively
^{b} Sonication energy of 0 Watt indicates that only vortexing and no sonication was used for preparing the first emulsion

Drug loading and encapsulation efficiency: Drug loading and drug encapsulation efficiency in AOT-alginate nanoparticles was dependent on AOT and alginate concentrations. Increasing the sodium alginate concentration from 0.1 to 1% w/v in the formulation resulted in an increase in methylene blue loading efficiency from 76.4±0.8 to 99.8±0.6% (Table 1). Similarly, increasing the AOT concentration from 0.05 to 20% in the formulation resulted in an increase in encapsulation efficiency from 16.7±0.8 to 99.8±0.6% (Table 2). These results could be explained based on the contribution of electrostatic interactions to drug loading in nanoparticles. Increasing the concentration of either alginate or AOT could result in greater electrostatic attraction between anionic alginate/AOT and weakly basic drug, resulting in better drug entrapment in nanoparticles.

In order to confirm the contribution of electrostatic interactions to drug encapsulation, the encapsulation of weakly acidic drugs, fluorescein sodium and diclofenac sodium, in nanoparticles was studied. Both diclofenac and fluorescein are low molecular weight drugs (Table 5), and are highly water-soluble. The encapsulation efficiency for fluorescein and diclofenac were low (˜6.0% and 6.2%, respectively; Table 5), suggesting that electrostatic interactions are an important determinant of drug encapsulation efficiency in AOT-alginate nanoparticles.

TABLE 5
Effect of drug used on loading and encapsulation efficiency ^{a}

| | Molecular | Drug | Encapsulation | Residual |
|---|---|---|---|---|
| | weight | loading | efficiency | methylene |
| Drug | (Da) | (% w/w) | (%) | chloride ^{b} |
| Rhodamine | 380 | 4.6 ± 0.2 | 59.7 ± 2.6 | 4 ppm |
| Doxorubicin | 580 | 3.8 ± 0.1 | 49.3 ± 1.5 | 3 ppm |
| Verapamil | 491 | 5.9 ± 0.5 | 76.8 ± 6.8 | 9 ppm |
| Clonidine | 266 | 3.6 ± 0.2 | 45.7 ± 1.9 | ND |
| Fluorescein | 332 | 0.6 ± 0.0 | 6.9 ± 0.2 | ND |
| Diclofenac | 318 | 0.5 ± 0.0 | 6.1 ± 0.4 | 2 ppm |

^{a} Sodium alginate and PVA concentrations were 1% w/v and 2% w/v, respectively. AOT concentration was 5% w/v and the phase volume was 1 mL.
^{b} ND—not determined

We also studied the effect of emulsification conditions (emulsifier concentration and energy input) on drug encapsulation efficiency. Increasing the PVA concentration in the external aqueous phase from 0.5 to 5% w/v resulted in an increase in drug encapsulation efficiency from 87.6±2.7% to 99.1±2.0% w/w (Table 3). Increasing the concentration of PVA in the external phase leads to increased viscosity of the external phase (see above) and higher amount of PVA adsorbed at the oil/water interface (Zambaux M. F. et al., J Control Release 50:31-40, 1998). This could lead to greater resistance to drug diffusion out of the oil phase and the consequent higher drug loading in nanoparticles. Similar effect of PVA on drug loading was observed with PLGA nanoparticles loaded with bovine serum albumin (Sahoo, S. K. et al., J Control Release 82:105-114, 2002). Increasing the energy input during the nanoparticle formulation resulted in a decrease in the drug encapsulation efficiency (Table 4). Drug encapsulation efficiency decreased from 99.8±0.6 to 75.5±0.4% when the sonication energy was increased from 18 Watt to 48 Watt. Decrease in emulsion droplet size with a consequent increase in the surface area available for drug loss may have contributed to the decrease in drug loading with increase in sonication energy.

Drug encapsulation efficiency in nanoparticles was also a function of the amount of drug added to the formulation as shown in Table 6. Encapsulation efficiency was 99.8±0.6% when 5 mg of methylene blue was used in nanoparticle formulation whereas the encapsulation efficiency decreased to 74.1±0.2% when 15 mg of methylene blue was used. To be effective, a delivery system should demonstrate high drug-loading capacity. As a reference, hydrophobic drugs like paclitaxel may be loaded in PLGA nanoparticles at ˜5% w/w drug loading (Sahoo, S. K. et al., Int. J. Cancer. 112:335-340, 2004).

TABLE 6
Effect of methylene blue amount added on particle size,
zeta potential and drug encapsulation ^{a}

| | Particle | Poly- | Drug | Encapsulation |
|---|---|---|---|---|
| Amount | size | dispersity | loading | efficiency |
| (mg) | (nm) | Index | (% w/w) | (%) |
| 5.0 | 241.6 ± 4.3 | 0.262 | 0.82 ± 0.01 | 99.8 ± 0.6 |
| 7.5 | 247.1 ± 0.7 | 0.238 | 0.67 ± 0.01 | 82.7 ± 1.1 |
| 10.0 | 267.0 ± 1.4 | 0.257 | 0.65 ± 0.01 | 80.2 ± 1.7 |
| 12.5 | 272.8 ± 1.8 | 0.217 | 0.63 ± 0.01 | 79.0 ± 0.7 |
| 15.0 | 292.8 ± 7.6 | 0.235 | 0.60 ± 0.01 | 74.1 ± 0.2 |

^{a} Alginate, AOT and PVA concentrations were 1% w/v, 20% w/v and 2% w/v, respectively

As shown in Tables 1, 3 and 6, drug loading in AOT-alginate nanoparticles varied between 0.5 to 0.8% w/w. Decreasing the AOT concentration in the formulation resulted in an increase in drug loading to ˜5% w/w; however, the drug encapsulation efficiency decreased with decrease in AOT concentrations (Table 2). In order to determine if higher amounts of drug may be loaded in nanoparticles without the loss of encapsulation efficiency, the volume of the AOT phase was decreased from 3 mL to 1.5 mL, without changing the concentration, in the emulsion used for preparing nanoparticles. This resulted in an increase in drug loading to about 1.9% w/v, with an encapsulation efficiency of 80% (Table 7). Decreasing the AOT concentration to 5% at this volume ratio further increased the drug loading to 3.8% w/w, with a drug encapsulation efficiency of ˜50%. The drug loading capacity of AOT-alginate nanoparticles is higher than that reported previously for other water-soluble drugs. For example, gelatin nanoparticles demonstrated a maximum of 3% w/w loading for methotrexate sodium (Cascone, M. G. et al., J Mater Sci Mater Med 13:523-526, 2002). PLGA nanoparticles showed 0.26% w/w loading for doxorubicin hydrochloride (Cascone, M. G. et al., J Mater Sci Mater Med 13:523-526, 2002). A maximum of 0.9% w/w loading was obtained for 5-fluorouracil in polycaprolactone nanoparticles (Cascone, M. G. et al., J Mater Sci Mater Med 13:523-526, 2002).

TABLE 7
Effect of AOT fraction on loading and encapsulation
efficiency of doxorubicin hydrochloride ^{a}

| Volume | AOT | Drug | Encapsulation |
|---|---|---|---|
| of AOT | Concentration | loading | efficiency |
| phase (ml) | (% w/v) | (% w/w) | (%) |
| 3 | 20 | 0.82 ± 0.01 | 99.8 ± 1.2 |
| 1.5 | 20 | 1.86 ± 0.01 | 80.0 ± 0.3 |
| 1 | 5 | 3.80 ± 0.11 | 49.3 ± 1.5 |

^{a} Sodium alginate and PVA concentrations were 1% w/v and 2% w/v, respectively

To confirm that AOT-alginate nanoparticles may be used for other weakly basic water-soluble drugs, the encapsulation efficiencies were investigated for other basic, water-soluble drugs such as verapamil, clonidine and doxorubicin hydrochloride. Because the above parameters (AOT concentration 5% and phase volume 1.5 mL) resulted in enhanced drug loading without compromising encapsulation efficiency, these parameters were used for encapsulating other drugs. Under similar formulation conditions, these drugs could be loaded in nanoparticles at similar drug loading and encapsulation efficiencies (Table 5). These studies further confirm the general applicability of AOT-alginate nanoparticles for weakly basic, low molecular weight, water-soluble drugs.

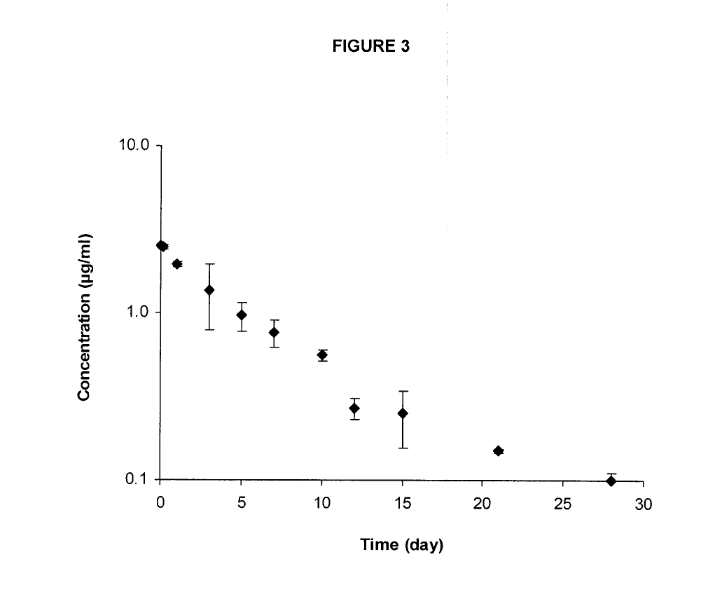

In vitro drug release studies: To determine the ability of AOT-alginate nanoparticles to sustain the release of hydrophilic drug, the in vitro release of verapamil, doxorubicin, clonidine and diclofenac from nanoparticles was studied. Initially, the stability of these drugs under the release conditions (PBS, pH 7.4 and 37° C.) were investigated. Verapamil, clonidine and diclofenac were stable under these conditions (data not shown), whereas doxorubicin demonstrated biphasic, first-order degradation profile (FIG. 3). Rate constants were determined for the two phases, and were used to correct the in vitro release of doxorubicin for degradation.

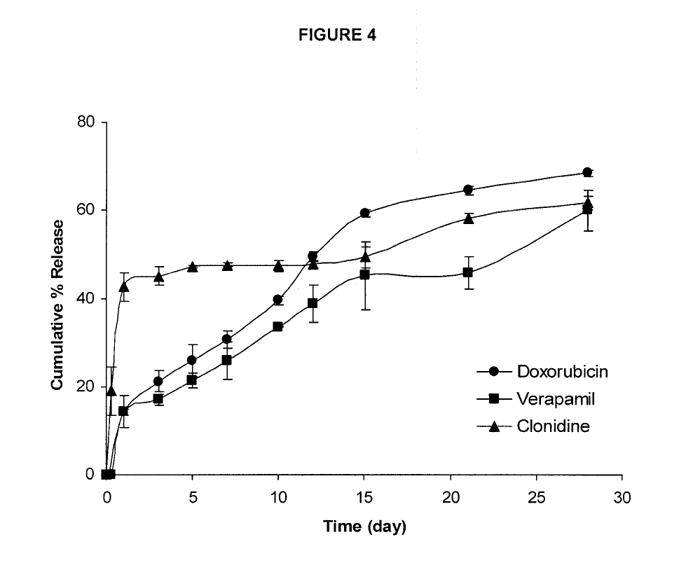

Nanoparticles demonstrated sustained drug release for all the three basic drugs investigated (FIG. 4). For both doxorubicin and verapamil, no drug release was observed during the first 8 hrs of the study. Following this lag period, the drug release was near zero-order (˜45 and 60% released; r² values of 0.9949 and 0.9977) in the first 15 days, followed by a more sustained drug release, with about 60-70% of the entrapped drug released over a 28-day period. In the case of clonidine, a burst release of about 19% was observed in the first 8 hrs, followed by a more sustained release (˜50%; r² values of 0.8820) over 15 days. About 62% of the encapsulated clonidine was released over a 28-day period.

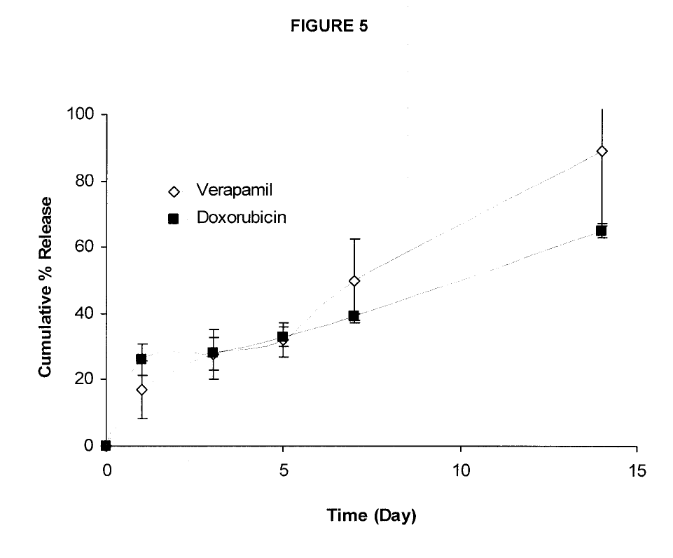

The possibility that AOT-alginate nanoparticles may be used to sustain the release of more than one drug was also investigated. Nanoparticles were loaded with 1.4% w/w of verapamil and 0.4% w/w of doxorubicin for this purpose. Doxorubicin, an anticancer agent, is a substrate of the drug efflux transporter P-glycoprotein while verapamil is a competitive inhibitor of P-glycoprotein. Thus, doxorubicin-verapamil combination could potentially be useful for treating drug-resistant cancers. In vitro release studies indicate that nanoparticles may simultaneously sustain the release of both drugs (FIG. 5). The release rate of the two drugs, however, was faster than from nanoparticles loaded with only one drug.

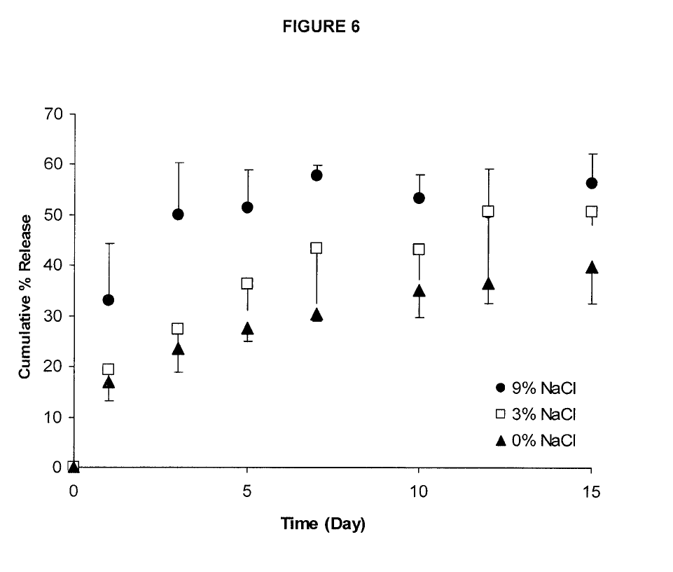

Previous studies with alginate delivery systems indicate that the main mechanism governing drug release in physiologic fluids is the sodium-calcium exchange. When calcium alginate is introduced in environment rich in monovalent salts (sodium, potassium), insoluble calcium alginate is converted into soluble sodium alginate, resulting in swelling, solubilization of the delivery system and drug release. In order to determine the contribution of sodium-calcium exchange to drug release, the effect of sodium ion concentration in the release medium on drug release was investigated. As shown in FIG. 6, increasing the concentration of sodium ions resulted in increase in rate and extent of drug release from nanoparticles. This strongly suggests that sodium-calcium exchange plays an important role in drug release from nanoparticles. Drug release in the absence of sodium ions suggest that other mechanisms such simple diffusion could also contribute to drug release. Because electrostatic interactions were found to be important for drug encapsulation, it was hypothesized that electrostatic interaction could also influence drug release from nanoparticles. If electrostatic interactions between drug and anionic matrix play a role in governing drug release, then, the release of weakly acidic drug from nanoparticles will be faster than that of a weakly basic drug.

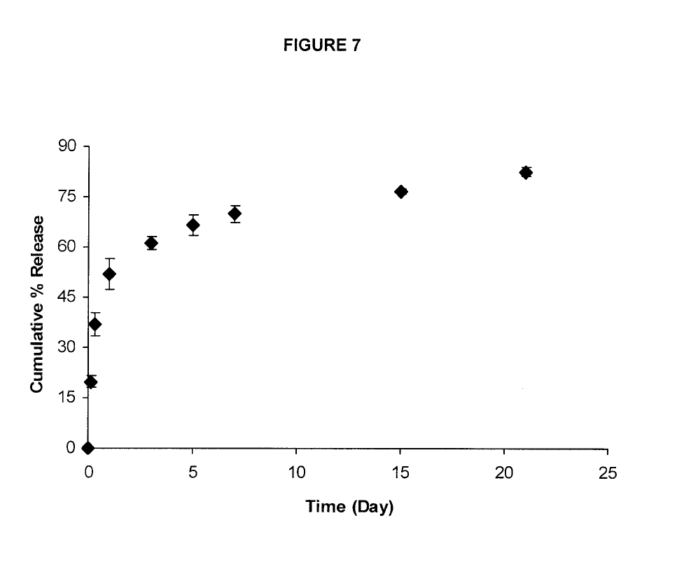

To this end, the release of a weakly acidic drug diclofenac from nanoparticles was investigated. As shown in FIG. 7, the release of diclofenac from nanoparticles was faster, with about 70% of the encapsulated drug released in 7 days. This may be compared to about 25-30% release observed for basic drugs in the same time frame. This study suggests that electrostatic interactions between drug and anionic nanoparticle matrix influence drug release from nanoparticles. The fact that increase in salt concentration in the release medium resulted in increased drug release from nanoparticles also points to the contribution of electrostatic interactions to drug release.

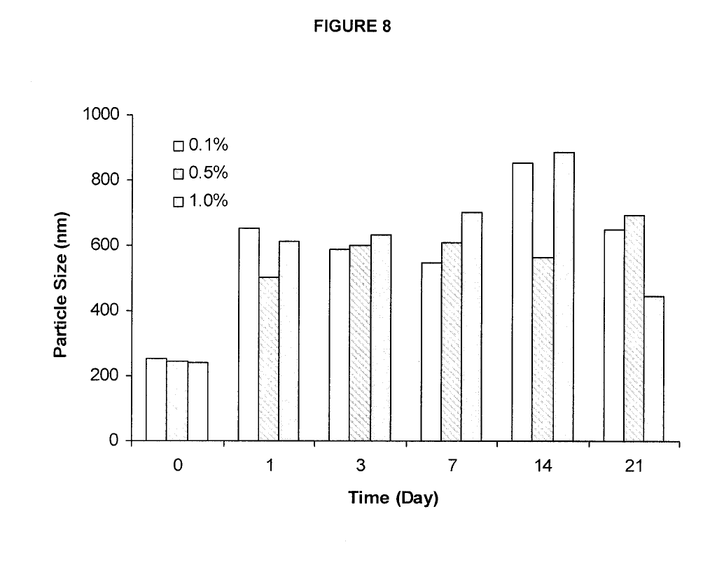

In order to clarify the effect of salt, the swelling kinetics of nanoparticles in PBS was studied. As discussed earlier, alginate systems swell in the presence of monovalent salts, due to conversion of calcium alginate to sodium alginate. Thus, if salt affected only electrostatic interactions without inducing calcium-sodium exchange, no swelling is expected. As shown in FIG. 8, there was significant swelling of nanoparticles in PBS. The size of nanoparticles increased from about 250 nm to about 500-600 nm on day 1 and to about 600-750 nm on days 14 and 21 (FIG. 8). After day 21, particle size could not be determined, probably due to disintegration of nanoparticles. It is possible that the observed increase in particle size could be due to aggregation of nanoparticles in solution over time. However, increase in particle size was qualitatively confirmed under a microscope, suggesting that AOT-alginate nanoparticles swell in buffer solutions. This further confirms that sodium-calcium exchange happens in AOT-alginate nanoparticles.

Basic drugs are encapsulated in nanoparticles through electrostatic interactions with the anionic components (AOT and alginate) of nanoparticles. The anionic functional groups (guluronic acid in alginate and sulfosuccinate group of AOT) also assist in crosslinking of nanoparticles with calcium. The in vitro release studies point to three possible mechanisms influencing drug release from nanoparticles. When nanoparticles come in contact with physiologic buffers, calcium in nanoparticles exchanges for sodium in the buffer. This results in swelling and slow dissolution of the delivery system and drug release. Presence of salt also favors reduced electrostatic interaction between the drug and nanoparticle matrix, resulting in release of the drug. As indicated by drug release in deionized water, drug release could also be mediated by mechanisms other than calcium-sodium exchange and electrostatic interactions. Calcium-sodium exchange, swelling and drug release have been described previously for other alginate systems (De, S. and Robinson, D., J. Control. Release 89:101-112, 2003). However, unlike other alginate systems, AOT-alginate nanoparticles do not rapidly disintegrate in physiological salt concentration, and were stable for more than 3 weeks. By introducing AOT, a molecule with highly electronegative sulfonate group, the rate of sodium-calcium exchange has been decreased and the release of basic drugs has been prolonged over a period of 4 weeks.

Although the release of acidic drugs like diclofenac from AOT-alginate nanoparticles was faster than that for basic drugs like verapamil, it has to be noted that the release was considerably sustained (70% release in 7 days) compared to other previously reported systems. For example, Yi and co-workers investigated alginate-bovine serum albumin nanoparticles for 5-fluorouracil (Yi, Y. M. et al., World J Gastroenterol 5:57-60, 1999). These nanoparticles released 84% of the encapsulated drug within 72 hours. Gelatin nanoparticles were investigated as carriers for methotrexate sodium (Cascone, M. G. et al., J Mater Sci Mater Med 13:523-526, 2002). The entire drug load was released within 150 hrs, with a burst release of 40% in the first 10 hrs. A surfactant-polymer system similar to AOT-alginate nanoparticles but composed of basic components (chitosan and a quaternary ammonium surfactant, for example) could be envisioned for acidic drugs. Such a system would be potentially useful for efficient encapsulation and sustained release of acidic drugs.

The following conclusions were drawn from this Example: Efficient encapsulation and sustained release of basic, water-soluble drugs from AOT-alginate nanoparticles has been demonstrated. Particle size of AOT-alginate nanoparticles was a function of emulsification conditions. Drug encapsulation efficiency was dependent on different formulation factors such as alginate, AOT, drug and PVA concentrations. Drug release from nanoparticles appeared to be mediated through sodium-calcium exchange as well as electrostatic interactions between drug and nanoparticle matrix. Sub-micron particle size and sustained release characteristics suggest that AOT-alginate nanoparticles are useful for sustained delivery of water-soluble drugs.

### Example 2

### Cellular Delivery of Water-Soluble Molecules

A novel surfactant-polymer nanoparticles for efficient encapsulation and sustained release of water-soluble drugs has been fabricated recently and disclosed in Example 1. These nanoparticles were formulated using aerosol OT (AOT; docusate sodium) and sodium alginate. AOT is an anionic surfactant that is approved as oral, topical and intramuscular excipient (U.S. Food and Drug Administration's Inactive Ingredients Database; www.accessdata.fda.gov). Sodium alginate is a naturally occurring polysaccharide polymer that has been extensively investigated for drug delivery and tissue engineering applications (Iskakov, R. M. et al., J. Control. Release 80:57-68, 2002; Shimizu, T. et al., Biomaterials 24:2309-16, 2003). The inventors have shown that AOT-alginate nanoparticles may sustain the release of water-soluble drugs such as doxorubicin and verapamil over a period of 4 weeks.

The objective of the instant example was to investigate the suitability of AOT-alginate nanoparticles as carriers for cellular delivery of water-soluble molecules. Using rhodamine and doxorubicin as model water-soluble molecules, the kinetics and mechanism of nanoparticle-mediated cellular drug delivery has been investigated.

### Materials and Methods

Materials: Rhodamine 123, sodium alginate, polyvinyl alcohol and calcium chloride were purchased from Sigma-Aldrich (St. Louis, Mo.). Aerosol OT, methanol and methylene chloride were purchased from Fisher Scientific (Chicago, Ill.).

Nanoparticle formulation: Nanoparticles were formulated by emulsification-crosslinking technology as described in Example 1. Sodium alginate solution in water (1.0% w/v; 1 mL) was emulsified into AOT solution in methylene chloride (20% w/v; 3 mL) by vortexing (Genie™, Fisher Scientific for 1 min over ice bath). The primary emulsion was further emulsified into 15 mL of aqueous PVA solution (2% w/v) by sonication for 1 min over ice bath to form a secondary water-in-oil-in-water emulsion. The emulsion was stirred using a magnetic stirrer, and 5 mL of aqueous calcium chloride solution (60% w/v) was added slowly to the above emulsion. The emulsion was stirred further at room temperature for ˜18 hrs to evaporate methylene chloride. For preparing drug-loaded nanoparticles, drug (5 mg) was dissolved in the aqueous alginate solution, which was then processed as above. Nanoparticles formed were recovered by ultracentrifugation (Beckman, Palo Alto, Calif.) at 145,000×g, washed two times with distilled water to remove excess PVA and unentrapped drug, resuspended in water, and lyophilized.

Determination of drug loading: Drug loading in nanoparticles was determined by extracting 5 mg of nanoparticles with 5 mL of methanol for 30 min and analyzing the methanol extract for drug content. Rhodamine and doxorubicin concentrations were determined by fluorescence spectroscopy (excitation/emission wavelengths of 485/528 nm; FLX 8000, Bio-Tek® Instruments, Winooski, Vt.). Drug loading was defined as the amount of drug encapsulated in 100 mg of nanoparticles, and represented as % w/w.

Determination of particle size and zeta potential: Particle size and zeta potential were determined using dynamic light scattering. Brookhaven 90Plus zeta potential equipment fitted with particle sizing software (Brookhaven instruments, Holtsville, N.Y.) was used. About 1 mg of nanoparticles was dispersed in 1 mL of distilled water by sonication, and was subjected to both particle size and zeta potential analysis.

In vitro release studies: Drug release from doxorubicin containing nanoparticles was determined in phosphate buffer saline (PBS, 0.15 M, pH 7.4) at 37° C. Nanoparticle suspension (1 mg/0.5 mL) was placed in dialysis chamber (MWCO 10,000 Da, Pierce), and the dialysis chamber was immersed in 10 mL of the release buffer in a 15-ml centrifuge tube. The centrifuge tube containing dialysis chamber was placed in an incubator shaker set at 100 rpm and 37° C. At predetermined time intervals, 0.5 mL of the release buffer was removed from the tube and was replaced with fresh release buffer. Doxorubicin concentration in the release buffer was determined by HPLC. A Beckman Coulter HPLC system with System Gold® 125 solvent module and System Gold® 508 autoinjector connected to Linear Fluor LC 305 fluorescence detector (Altech) set at 505/550 nm wavelengths were used. A Beckman® C-18 (Ultrasphere) column (ODS 4.6×250 MM) was used. Acetonitrile: water (adjusted to pH 3 with glacial acetic acid) (30:70) was used as mobile phase at a flow rate of 1 mL/minute. Retention time of doxorubicin was 7 minutes.

Cell culture: Human breast cancer cells (MDA-Kb2 and MCF-7) were used as model cell lines. MDA-Kb2 cells were cultured in Leibovitz's medium supplemented with 10% FBS at 37° C. MCF-7 cells were grown in RPMI medium supplemented with 10% FBS at 37° C. and 5% CO₂.

Cellular uptake of nanoparticles: Nanoparticles containing rhodamine were used for the study. All the studies were performed at 37° C., unless otherwise specified. MDA-kb2 cells were seeded in a 24-well plate at a density of 50,000 cells/well and allowed to attach overnight. Cells were then treated with nanoparticle suspension in complete growth medium. To determine the effect of dose of nanoparticles on uptake, cells were treated with various doses (12.5 to 200 μg/mL) of nanoparticles for 2 hrs. To determine the effect of time of treatment, cells were treated with constant dose (100 μg/mL) of nanoparticles for varying periods of time (30 to 120 min). At the end of the treatment period, the cell monolayer was washed three times with cold PBS. Cells were then lysed using 100 μl of 1× cell culture lysis reagent (Promega).

The protein content of the cell lysate was determined using the Pierce BCA protein assay (Rockford, Ill.). Cell lysates were then analyzed for rhodamine content. To study the effect of metabolic inhibition on nanoparticle uptake, cells were preincubated with growth medium containing 0.1% w/v sodium azide and 50 mM deoxyglucose for 1 hr, and then incubated with nanoparticle suspension (100 μg/mL) containing 0.1% w/v sodium azide and 50 mM of deoxyglucose for 2 hrs. To study the effect of temperature on cellular uptake of nanoparticles, cells were preincubated at 4° C. for 1 hr and then treated with the nanoparticle suspension (100 μg/mL) at 4° C. for 2 hrs.

Exocytosis of nanoparticles: A previously reported exocytosis assay was used (Panyam J. and Labhasetwar V., Pharm Res 20:212-20, 2003). In brief, cells were incubated with nanoparticles (100 μg/mL) for 2 hrs in growth medium, followed by washing with PBS twice. The intracellular nanoparticle concentration at the end of the 2-hr incubation period was taken as the zero time point value. Cells were then incubated with fresh growth medium. At different time intervals, medium was removed; cells were washed twice with PBS and lysed as described above. Rhodamine concentration in the cell lysate was determined as described below. Data was represented as the percent of nanoparticles that were retained at different time intervals relative to the zero time point value.

Quantification of rhodamine in cell lysates: Cell lysates were mixed with 300 μL of methanol and incubated at 37° C. for 6 hrs at 100 rpm. The samples were centrifuged at 14,000 rpm for 10 min at 4° C. Rhodamine-associated fluorescence in the supernatants was determined using a microplate reader as described for drug loading determination. Data was expressed as rhodamine accumulation normalized to total cell protein.

In vitro cytotoxicity with doxorubicin-loaded nanoparticles: MCF-7 cells were plated in 96-well plates at 5,000 cells/well/0.1 mL medium. On Day 0, cells were treated with either 0.5 or 0.75 μM doxorubicin in solution or encapsulated in nanoparticles. Untreated cells and blank nanoparticle-treated cells were used as controls for solution-treated and nanoparticles-treated cells, respectively. On Day 2, cells were washed to remove the treatments and added with fresh medium. Medium was changed every other day with no fresh dose of the treatments added. Cytotoxicity was determined at different time points using MTS assay (CellTiter 96 AQueous, Promega). Cytotoxicity was determined as a percent of respective controls.

The following results were obtained from the experiments of this Example.

Nanoparticle characterization: Nanoparticles were initially characterized for particle size, polydispersity, zeta potential, and drug loading. As shown in Table 8, both rhodamine-loaded nanoparticles and doxorubicin-loaded nanoparticles had sub-micron particle size (500-700 nm) and polydispersity index (˜0.28). The zeta potential of nanoparticles was around −13 to 14 mV. Both rhodamine and doxorubicin could be efficiently encapsulated in nanoparticles (4.6% drug loading for rhodamine and 3.8% for doxorubicin). Nanoparticles were stable to lyophilization and in various buffers and cell culture medium. Nanoparticles did not aggregate in the presence of serum.

TABLE 8
AOT-alginate nanoparticles loaded with rhodamine or doxorubicin

| | z-Average | Poly- | | Drug |
|---|---|---|---|---|
| | particle size | dispersity | Zeta potential | loading |
| Drug | (nm) | index | (mV) | (mg/100 mg) |
| Rhodamine | 515 | 0.284 | −14.6 ± 2.1 | 4.6 ± 0.2 |
| Doxorubicin | 689 | 0.286 | −13.4 ± 1.0 | 3.8 ± 0.1 |

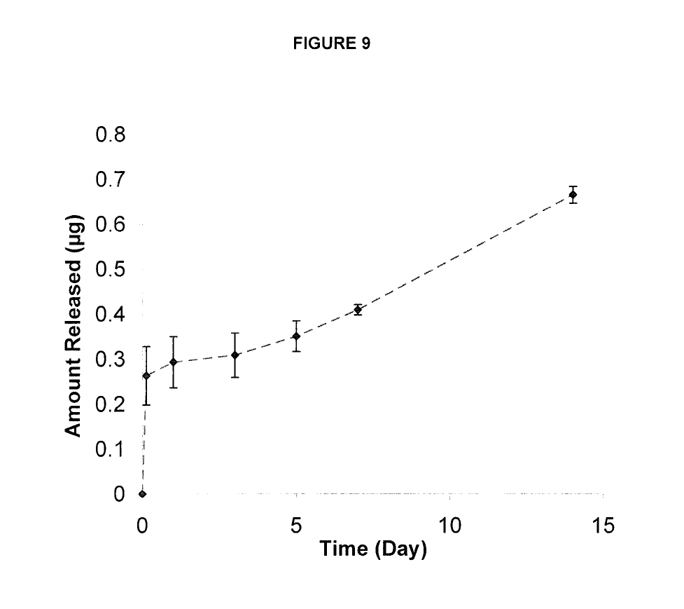

In vitro drug release: In vitro release studies under sink conditions in phosphate buffered saline (pH 7.4, 0.15 M) indicated that nanoparticles released about 59.2±0.8% of the entrapped drug over a period of 15 days (FIG. 9). The drug release was linear (r2=0.895), suggesting a zero-order drug release. In this time period, nanoparticles released doxorubicin at the rate of 2.3 μg/day/mg nanoparticles.

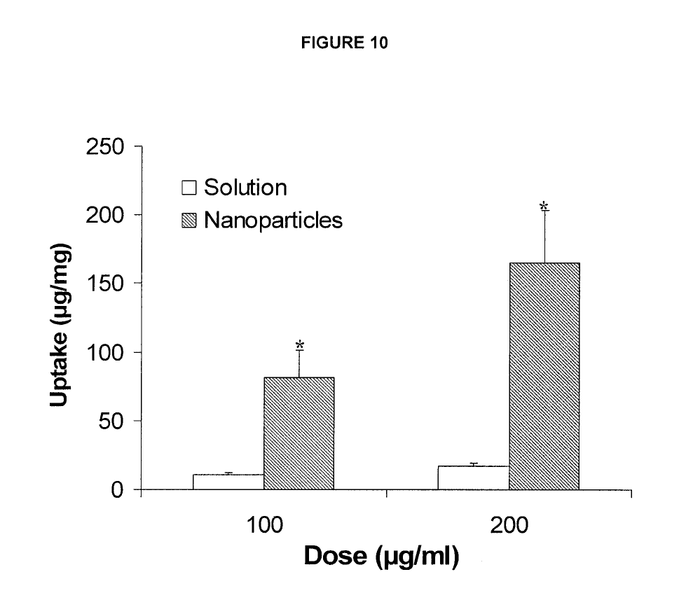

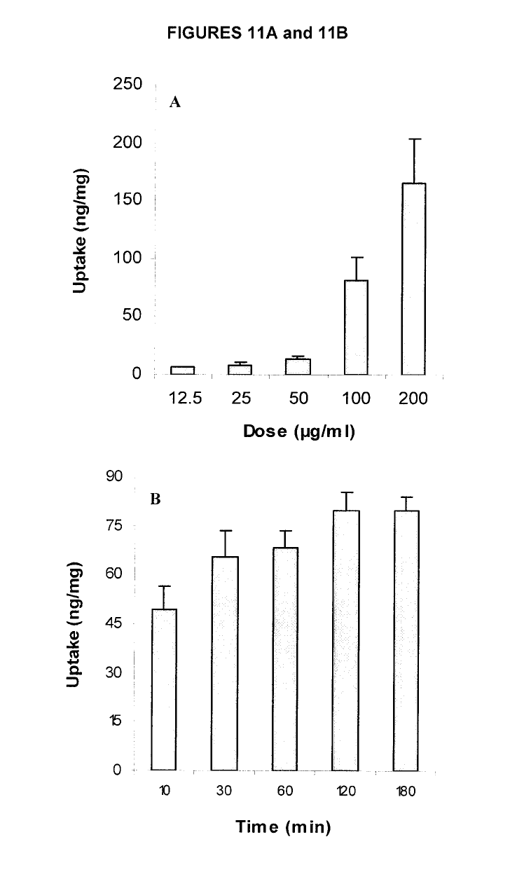

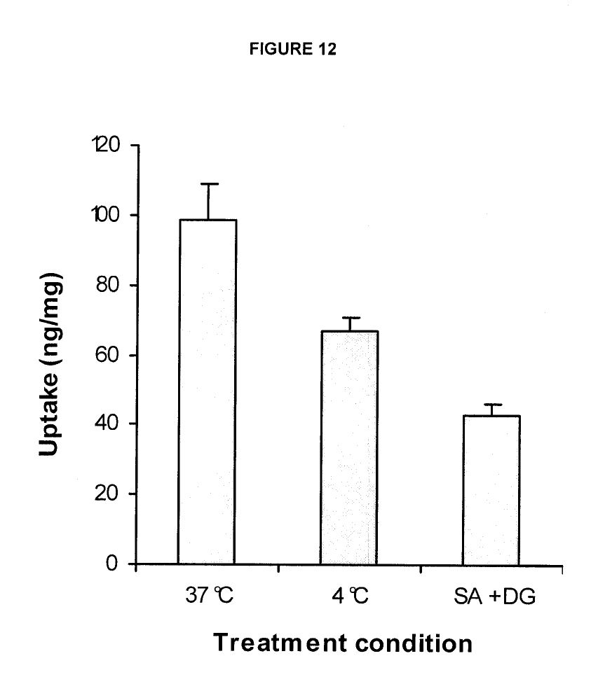

Kinetics and mechanism of nanoparticle uptake: To determine the efficacy of cellular drug delivery with AOT-alginate nanoparticles, the cellular accumulation of rhodamine following treatment with rhodamine in solution or in nanoparticles was compared. As shown in FIG. 10, treatment with rhodamine in nanoparticles resulted in a 7.5- to 10-fold higher accumulation of rhodamine than with rhodamine in solution. The increase in rhodamine accumulation with nanoparticles was significant (p<0.05) and dose-dependent. Further, the kinetics of cellular rhodamine accumulation with nanoparticles was studied. Rhodamine accumulation into cells with nanoparticles was both dose- and time-dependent (FIG. 11). Rhodamine accumulation increased proportionately with dose at lower doses (up to 50 μg/mL dose), but was disproportionate at higher doses. Also, nanoparticle uptake into the cells increased with time of incubation, reaching a steady state at about 90 min. In order to determine the mechanism of nanoparticle uptake into cells, the energy dependence of nanoparticle uptake in cells was evaluated. Reducing the cellular ATP production by incubating cells with metabolic inhibitors sodium azide and deoxyglucose resulted in ˜50% reduction in cellular uptake of nanoparticles (FIG. 12). Decreasing active processes in cells by incubating cells at 4° C. had a similar effect on nanoparticle uptake into cells (FIG. 12). Energy dependence of nanoparticle uptake, along with dose- and time-dependence, suggests that nanoparticle uptake into the cells is an endocytic process.

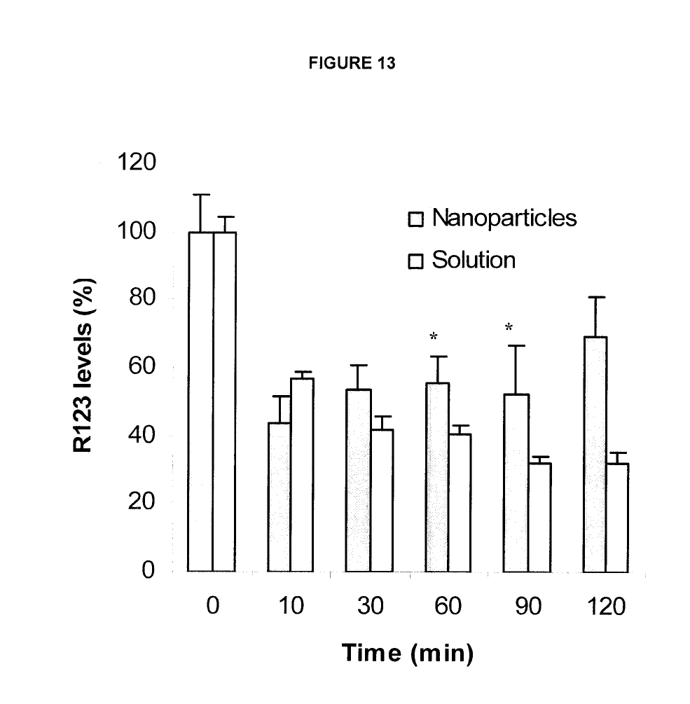

Exocytosis and retention of nanoparticles: As indicated in FIG. 11, continuous incubation of cells with nanoparticles resulted in an increase in drug accumulation, followed by steady state cellular levels. However, when cells were washed off of nanoparticles following initial incubation, intracellular levels began to decline. Previous studies have shown that this decline is due to the exocytosis of the delivery system from the cells (Sahoo, S. K. and Labhasetwar, V., Mol Pharm 2:373-83, 2005; Panyam, J. and Labhasetwar, V., Pharm Res 20:212-20, 2003). As shown in FIG. 13, exocytosis of AOT-alginate nanoparticles was relatively rapid immediately after the treatment was removed; about 50% of the internalized particles exited in 10 min. Cellular levels of rhodamine remained steady beyond 10 min. Cellular retention of the drug following treatment with drug in solution was significantly less than that with drug in nanoparticles. At the end of 120 min, there was almost a 2-fold difference in between the two treatments in the fraction of internalized drug retained within the cells. Also, the drop in cellular drug levels following treatment with drug in solution was biphasic; an initial rapid drop immediately following the removal of the treatment, followed by a much slower rate of decrease beyond 10 min.

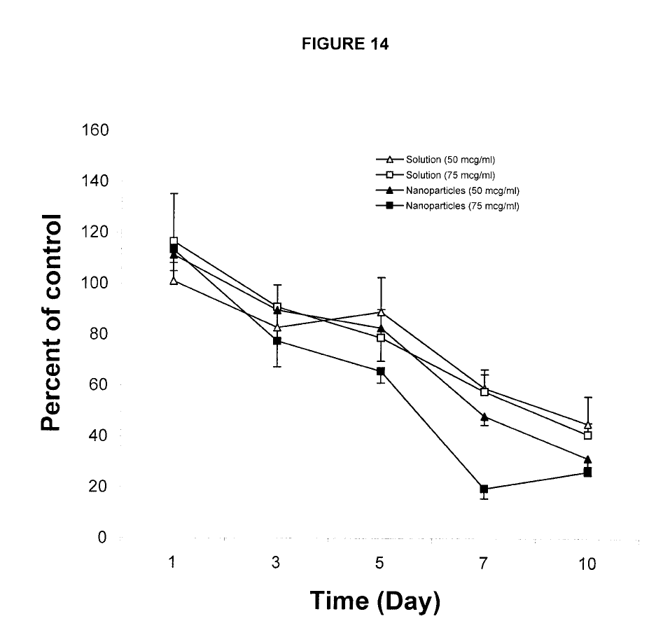

Cytotoxicity of doxorubicin-loaded nanoparticles: In order to determine the therapeutic efficacy of nanoparticle-encapsulated drug, the cytotoxicity of nanoparticle-encapsulated doxorubicin in vitro was evaluated. Doxorubicin in nanoparticles demonstrated significantly higher cytotoxicity than doxorubicin in solution (FIG. 14). This enhancement in cytotoxicity with nanoparticles was dose-responsive and was sustained for the 10 days of study. There was no significant difference in the viability of untreated cells and cells treated with blank nanoparticles, indicating that at the concentration tested, blank nanoparticles were not toxic to cells.

Nanoparticle-mediated cellular drug delivery is governed by the dynamics of cellular uptake and retention of nanoparticles (Sahoo, S. K. and Labhasetwar, V., Mol Pharm 2:373-83, 2005; Panyam J and Labhasetwar V, Pharm Res 20:212-20, 2003) and the rate of drug release from nanoparticles (Panyam, J. and Labhasetwar, V., Mol Pharm 1:77-84, 2004). Previous studies demonstrate that uptake and retention of drug carriers like nanoparticles are affected by cellular processes such as endocytosis and exocytosis (Panyam, J. and Labhasetwar, V., Pharm Res 20:212-20, 2003). These cellular processes are, in turn, influenced by nanoparticle properties such as particle size and zeta potential (Desai, M. P. et al., Pharm Res 13:1838-45, 1996; Desai, M. P. et al., Pharm Res 14:1568-73, 1997; Sahoo, S. K. et al., J Control Release 82:105-14, 2002).

AOT-alginate nanoparticles investigated in this study are useful for efficient encapsulation and sustained release of water-soluble drugs like doxorubicin. In vitro release studies show that nanoparticles result in a near zero-order release of doxorubicin over a 15-day period. Example 1 demonstrated that electrostatic interactions between weakly basic drug and anionic nanoparticle matrix composed of alginate and AOT contribute to the efficient encapsulation and sustained drug release properties of AOT-alginate nanoparticles. Following encapsulation of weakly basic drugs, nanoparticles have a net negative charge, which stabilizes nanoparticles in buffer and in medium containing serum. This is an advantage over other nanoparticle delivery systems such as polycyanoacrylate nanoparticles that become cationic following encapsulation of weakly basic drugs like doxorubicin (Brigger I. et al., J Control Release 100:29-40, 2004).

Nanoparticles resulted in significantly higher cellular drug accumulation than drug in solution. Weak bases such as rhodamine and doxorubicin are positively charged at physiologic pH (Martin, A. et al., Physical pharmacy. Physical chemical principles in the pharmaceutical sciences, Waverly International, Baltimore, 1993). For example, doxorubicin, which has a pKa of ˜8.2 (Scholtz, J. M., Antineoplastic drugs. In Beringer P. et al. (eds), Remington: The science and practice of pharmacy Lippincott Williams and Wilkins, Philadelphia, 2000, pp. 1556-1587), is about 86% ionized at pH 7.4. Because the cell membrane is lipophilic and limits the diffusion of compounds that are ionized, availability of doxorubicin at its intracellular site of action is limited (Franklin, M. R. and Franz, D. N., Drug absorption, action, and disposition. In P. Beringer P. et al. (eds), Remington: The science and practice of pharmacy Lippincott Williams and Wilkins, Philadelphia, 2000, pp. 1142-1170). Higher drug accumulation with nanoparticles than with solution suggests that processes other than simple diffusion are involved in nanoparticle-mediated cellular drug delivery. Previous studies have shown that nanoparticles formulated using polymers such PLGA are taken up into cells through active process such as endocytosis (Panyam, J., et al., Faseb J 16:1217-26, 2002). Energy dependence of nanoparticle uptake into cells suggests that cellular uptake of AOT-alginate nanoparticles involves endocytosis (Mukherjee, S. et al., Physiol Rev 77:759-803, 1997). This is further confirmed by the achievement of steady state in drug accumulation with prolonged incubation time. Because endocytosis is an active process and is limited by the number of endocytic vesicles originating from the cell membrane, drug accumulation involving endocytosis eventually reaches steady state.

Retention studies suggest that a fraction of internalized nanoparticles come out of the cell following the removal of nanoparticles from the external media. This exocytosis process has been observed for other delivery systems including liposomes (Colin, M. et al., Gene Ther. 7:139-152, 2000) and nanoparticles (Panyam, J. and Labhasetwar, V., Pharm Res 20:212-20, 2003). Exocytosis is a process by which cells release cellular signals and expel waste into the external environment (Greenwalt, T. J., Transfusion 46:143-52, 2006; Pickett, J. A. and Edwardson, J. M., Traffic 7:109-16, 2006). The current model for endocytosis and exocytosis suggests the existence of three different cellular compartments in the endocytosis/exocytosis pathway (Gruenberg, J., Nat. Rev. Mol. Cell Biol. 2:721-730, 2001). Cells internalize external materials through early endocytic vesicles (early endosomes), which are then trafficked to sorting endosomes. Sorting endosomes sort the incoming materials. Depending on the signals present in the incoming molecules, they are recycled back to the outside of the cell through recycling endosomes, diverted to other cellular organelles such as endoplasmic reticulum, or forwarded to lysosomes for degradation. Differences in the kinetics of drug loss from the cells following treatment with drug in solution and drug in nanoparticles suggest that different processes may be involved in drug loss from cells. Simple diffusion out of the cell could be responsible for drug loss following treatment with drug solution, whereas exocytosis may be involved in the case of drug in nanoparticles (Panyam, J. and Labhasetwar, V., Pharm Res 20:212-20, 2003).

Enhanced accumulation and sustained cellular retention of the drug following treatment with nanoparticles, suggests that nanoparticles may enhance the efficacy of drugs whose site of action is intracellular. Doxorubicin was used as a model drug to study therapeutic efficacy, because doxorubicin causes cytotoxicity by intercalation with DNA in the nucleus. As expected, doxorubicin in nanoparticles was significantly more cytotoxic than doxorubicin in solution, thus, confirming the potential of nanoparticles for enhanced and sustained cellular drug delivery. Enhanced uptake and sustained release of nanoparticle-encapsulated doxorubicin within the cells could be responsible for the sustained enhancement of cytotoxicity observed with nanoparticle-encapsulated doxorubicin.

The results described in Example 2 show that AOT-alginate nanoparticles significantly enhanced and sustained the cellular delivery of basic, water-soluble drugs. This translates into enhanced therapeutic efficacy for drugs like doxorubicin that have intracellular site of action. Based on these results, it can be concluded that AOT-alginate nanoparticles are suitable carriers for enhanced and sustained cellular delivery of basic, water-soluble drugs.

### Example 3

### Enhancing Chemo- and Photodynamic Therapy in Breast Cancer Using Nanotechnology

This Example was performed to test the in vivo and in vitro efficacy of nanoparticle-mediated combination chemo- and photodynamic therapy in a mouse model of drug-resistant tumor. Drug-resistant JC tumors (doxorubicin-resistant mammary adenocarcinoma) grown subcutaneously in female Balb/c mice were used in the studies. As discussed below, combination treatment with nanoparticle-conjugated doxorubicin and photodynamic therapy significantly enhanced tumor inhibitory property. These findings indicate that tumors responsive to combination therapy contain infiltrating immune cells with lymphocytic morphology. The Example also demonstrates reduced tumor cell proliferation and fewer angiogenic blood vessels in treated tumors than in untreated tumors. In vitro studies on a human chemoresistant breast cancer cell line have shown that nanoparticle-mediated photodynamic therapy effectively sensitizes these cells to chemotherapy.

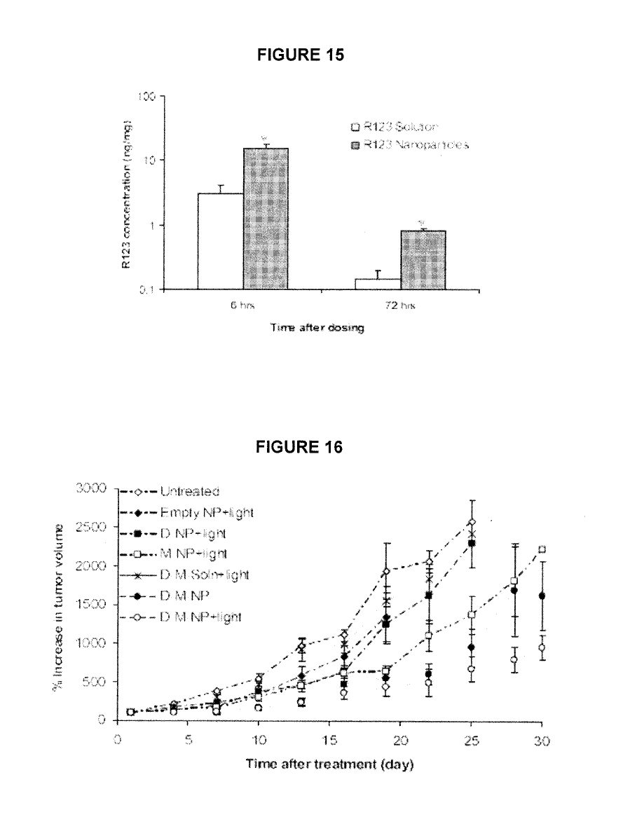

One objective of this Example was to determine the ability of AOT-alginate nanoparticles to enhance the tumor accumulation of encapsulated rhodamine 123. Drug-resistant JC tumors grown subcutaneously in Balb/c mice were used in the study. Rhodamine in solution or an equivalent dose encapsulated in nanoparticles was injected intravenously through the tail vein. As can be seen in FIG. 15, encapsulation in nanoparticles resulted in a significant and sustained increase in the amount of rhodamine delivered to the target tumor tissue (˜5-fold at 6 hrs and 72 hrs; P<0.05 for both time points). Previous studies showed that nanoparticulate carriers can increase tumor-specific accumulation of encapsulated drug through ‘Enhanced Permeation and Retention’ effect. Tumors, because of their leaky vasculature, allow enhanced accumulation of colloidal carriers such as nanoparticles. Because tumors have poor lymphatic drainage, nanoparticles are trapped within the tumor tissue.

Nanoparticle-mediated combination PDT-chemotherapy inhibited drug-resistant tumor growth. The in vivo efficacy of nanoparticle-mediated combination chemo- and photodynamic therapy was studied in a mouse model of drug-resistant tumor. Drug-resistant JC tumors (doxorubicin-resistant mammary adenocarcinoma) grown subcutaneously in female Balb/c mice were used in these experiments. Mice were administered a single i.v. dose of the different treatments. Doxorubicin treatment did not show a significant therapeutic effect. Mice treated with combination therapy nanoparticles along with light activation showed a significant inhibition of tumor growth (P<0.05), compared to those treated with doxorubicin nanoparticles or other controls (FIG. 16). In addition, treatment with combination therapy without light exposure also resulted in significant tumor inhibition compared to other controls. This is consistent with the observation that methylene blue can increase doxorubicin efficacy independent of its PDT efficacy. This Example demonstrates the superior efficacy of nanoparticle-mediated combination therapy against drug-resistant tumor. As shown in FIG. 16, nanoparticle-mediated combination PDT and chemotherapy overcame tumor drug resistance in vivo. Female Balb/c mice bearing JC tumors of at least 100 mm³ volume were injected intravenously with treatments equivalent to 8 mg/kg dose of methylene blue and 4 mg/kg doxorubicin. About 24 hrs after treatment administration, tumors were exposed to light of 665 nm wavelength (50 J/cm²). Animals were then monitored for tumor growth.

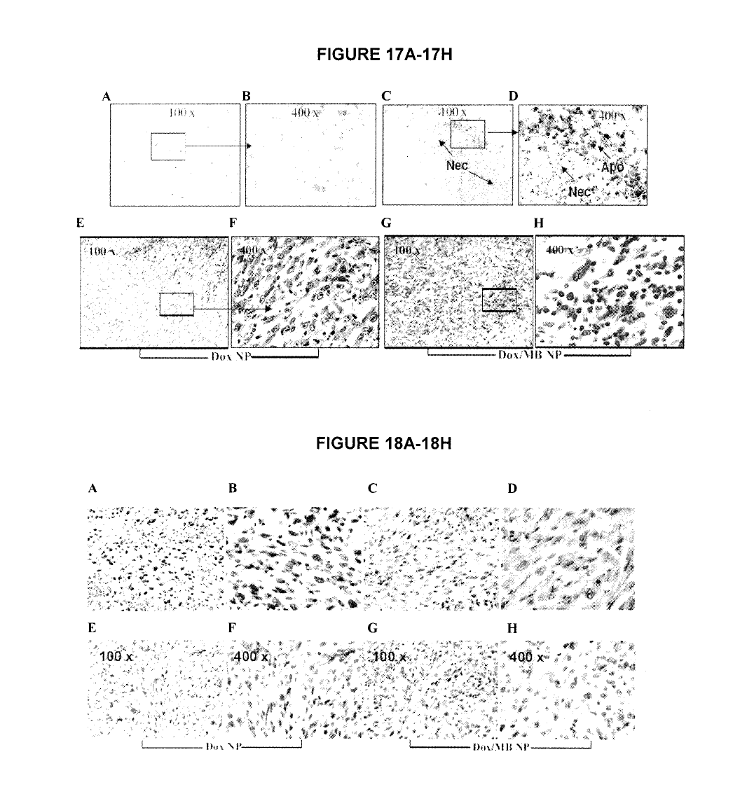

Nanoparticle-mediated combination therapy induced necrosis and immune cell recruitment. The objective was to investigate the mechanism of tumor inhibition with combination therapy in a mouse model of drug-resistant cancer. Induction of apoptosis/necrosis was determined by TUNEL assay while recruitment of immune cells into tumors was determined by histology. As indicated in FIG. 17, combination therapy resulted in significant apoptosis and necrosis, whereas chemotherapy did not induce significant necrosis. Induction of necrosis is important, because necrosis is an initiating event for immune response against the tumor tissue. FIG. 17 also shows the infiltration of immune cells in specific regions of tumors that were treated with combination therapy. Densely stained nucleus with little cytoplasm suggests a lymphocyte morphology.

This Example also shows that nanoparticle-mediated combination therapy inhibited tumor cell proliferation. The mechanism of tumor inhibition with combination therapy was studied in a mouse model of drug-resistant cancer. Tumor cell proliferation was evaluated by determining PCNA expression. As indicated in FIG. 18, combination therapy resulted in a significant decrease in PCNA expression, suggesting reduced tumor cell proliferation. In addition, the effect of combination therapy on angiogenesis was evaluated. Tumor tissues were stained for CD34 positive endothelial cells as a marker for angiogenesis. FIG. 18 shows that there was not only a decrease in number of CD34 positive vessels in treated as compared to controls but also that the CD34 positive vessels were defective as displayed by very weak CD34 staining intensity. Further, as compared to controls, where CD34+ vessels were well-defined, very diffuse vessels were present in treated tumors.

### Example 4

### Photodynamic Therapy (PDT) as a Treatment Modality for Cancer

Methylene blue, sodium alginate, polyvinyl alcohol and calcium chloride were obtained from Sigma-Aldrich (St. Louis, Mo.). Aerosol OT, methanol and methylene chloride were obtained from Fisher Scientific (Chicago, Ill.). 3′-(p-aminophenyl)fluorescein (APF) was obtained from Invitrogen (Carlsbad, Calif.). CellTiter 96® AQᵤₑₒᵤₛ was obtained from Promega (Madison, Wis.). Nanoparticles were formulated by a multiple-emulsion solvent evaporation cross-linking technique. Chavanpatil M, et al. Polymer-surfactant nanoparticles for sustained release of water-soluble drugs. J Pharm Sci 2007; In Press.

Briefly, an aqueous solution of sodium alginate (sodium alginate 1.0% w/v; 1 ml) was emulsified into AOT in methylene chloride (2.5% w/v; 2 ml) by sonication (Sonabox™, Misonix, Inc.) for 1 minute over an ice bath. The w/o emulsion was further emulsified into an aqueous solution of polyvinyl alcohol (PVA) (2% w/v; 15 ml) by sonication for 1 minute over an ice bath to form w/o/w emulsion. Five ml of aqueous solution of calcium chloride (60% w/v) was gradually added to the emulsion with gentle stirring. Methylene chloride was evaporated by over night gentle stirring at room temperature then for 1 hour under vacuum. To prepare methylene blue loaded nanoparticles, 5 mg of methylene blue was dissolved in the aqueous solution of sodium alginate then processed as described above. Nanoparticles were collected by ultracentrifugation for 30 minutes at 145,000×g for 3 cycles (Beckman, Palo Alto, Calif.) washing in between with deionized water. Dry nanoparticles were recovered by lyophilization (FreeZone 4.5®, Labconco Corp., Kansas City, Mo.).

Particle size was measured using Atomic Force Microscopy (AFM) in the tapping mode. For AFM, silicon tapping tips (TESP, VEECO) were used with a nominal tip radius less than 10 nm as provided by the manufacturer. Briefly, a droplet of an aqueous suspension of nanoparticles (100 μg/ml) was spread over a thin layer of polyethyleneimine-coated glass coverslip then air dried. Nanoparticles were then imaged using Nanoscope III (Digital Instruments/VEECO) with an E scanner (maximum scan area=14.2×14.2 μm2). The scan rate was 1 Hz and the integral and proportional gains were approximately 0.4 and 0.7, respectively. Heights images were plane-fit in the fast scan direction with no additional image filtering.

Zeta potential and polydispersity were determined using dynamic light scattering. Briefly, 1 mg of nanoparticles was suspended in 1 ml deionized water by sonication then subjected to zeta potential analysis using Brookhaven 90Plus zeta potential equipment.

Methylene blue loading in nanoparticles was determined by extracting 5 mg of nanoparticles in 5 ml of methanol for 1 hour in dark at room temperature. Methylene blue concentration in the methanolic extract was determined by using HPLC. Beckman Coulter HPLC system with System Gold® 125 solvent module and System Gold® 508 auto-injector connected to System Gold® 168 PDA detector were used. Beckman® C-18 (Ultrasphere) column (ODS 4.6×250 MM) and UV detection at 598 nm wavelength were used. Acetonitrile; ammonium acetate (10 mM, pH 4 adjusted with glacial acetic acid) was used as mobile phase at 1 ml/minute flow rate. Retention time was ˜8 minutes. Drug loading in nanoparticles (w/w) was defined as the amount of methylene blue (mg) in 100 mg nanoparticles.

For cytoxicity studies, MCF-7 cells were allowed to attach in 96-well plates (5,000 cells/well/0.1 ml) for 24 hours. On the day of the treatment, medium was removed and cells were incubated with medium containing either 0.3 or 0.6 μM methylene blue in solution or encapsulated in nanoparticles. Untreated cells and cells treated with an equivalent amount of blank nanoparticles were used as controls. After one hour, treatments were removed, cells were washed twice with PBS and fresh medium was added. Cells were photo-irradiated with different doses of light at 665 nm wavelength (LumaCare™ LC-122M, Newport Beach, Calif.). Cells that received same treatments as above without light-irradiation were used as negative controls. Cytotoxicity was determined using commercially available cytotoxicity assay (CellTiter 96® AQᵤₑₒᵤₛ, Promega).

MCF-7 cells were allowed to attach in 24-well plates (50,000 cells/well/ml) for 24 hours. Cells were then treated with 0.3 μM methylene blue in solution or encapsulated in nanoparticles. After 1 hour, treatments were removed and cells were washed twice with PBS. Cells were lysed using cell lysis buffer (1% Triton-X 100 in 0.1 M phosphate buffer, pH 6.5; 300 μl/well) and incubation in orbital incubator shaker (Brunswick Scientific, C24 incubator shaker, NJ) for one hour at 100 rpm and 37° C. Protein content of the cell lysate was determined using BCA Peirce protein assay reagents (Rockford, Ill.). Methylene blue was extracted from cell lysate with 1 ml methanol and methylene blue concentration was analyzed using LC-MS. A Waters Alliance® HT 2795 HPLC system (Waters®, Milford, Mass.) with an autosampler was used. A Synergi® Polar-RP (4 micron, 150×4.6 mm) column was used (Phenomenex, Torrance, Calif.). Acetonitrile: 10 mM ammonium acetate buffer (adjusted to pH 4 with glacial acetic acid) (78:22) was used as mobile phase at a flow rate of 1.4 ml/min. Eluted MB (˜9 minutes) was monitored at 284.1 molecular mass using Waters' ZQ2000 single quadrupole mass spectrometer.

### Nanoparticle-Mediated ROS Generation Ex Vitro

To study the effect of encapsulation in nanoparticles on the ROS yield, methylene blue in solution or in nanoparticles (0.3 or 0.6 μM in PBS) was photo-activated in the presence of 10 μM 3′-(p-aminophenyl)fluorescein (APF), with a measured dose of light (1200 mJ/cm²) using a light source of 665 nm wavelength. Fluorescein generated was determined by measuring increasing fluorescence using fluorescence spectroscopy (excitation/emission wavelengths of 485/528 nm; FLX 8000, Bio-Tek® Instruments, Winooski, Vt.). PBS and empty nanoparticles were used as negative controls. To determine the effect of dose of light on the amount of ROS generated, above samples were photo-activated with 10 consecutive doses of light (1200 mJ/cm² per dose) measuring fluorescence after each illumination. To determine the effect of inactive components of nanoparticles on generation of ROS in general, free methylene blue was mixed with empty nanoparticles and treated as above. Experiments performed as above but without light irradiation were used as light negative controls.

### Nanoparticle Characterization

Nanoparticles were characterized for morphology, particle size, polydispersity, zeta potential and drug loading. Particles' morphology and number-average size were determined using Atomic Force Microscopy (AFM). Nanoparticles size was measured using Nanoscope 5.12b48 software and was around 72±11 nm. Zeta potential and polydispersity index were around −19.33±1.25 mV and 0.3, respectively. Methylene blue was efficiently encapsulated in the nanoparticles (90.0% w/w).

### Cytotoxicity Studies

In order to determine the effect of encapsulation in nanoparticles on PDT of methylene blue, the cytotoxicity of nanoparticle-mediated PDT was evaluated in MCF-7 cells. Photo-activated methylene blue in nanoparticles showed a significantly higher cytotoxicity than methylene blue in solution. The enhanced cytotoxicity with nanoparticles was dose-responsive (0.3 vs. 0.6 μM) and sustained over a period of 7 days. Untreated cells and cells treated with empty nanoparticles then light-activated showed no significant cytotoxicity indicating that blank nanoparticles do not cause cytotoxicity and/or photodynamic effect. Cells received same treatments as above without light-activation showed no significant effect.

In order to determine the effect of dose of light on nanoparticle-mediated PDT, MCF-7 cells were treated with 0.3 μM then received different doses of light (480, 1200 or 2400 mJ/cm²). Photo-activation of methylene blue in nanoparticles with increasing doses of light resulted in significant and increased cytotoxicity indicating that PDT with nanoparticles was responsive to the dose of light. Further, MB in nanoparticles was significantly more effective than that in solution at all the doses of light.

### Cellular Accumulation

To evaluate the effect of nanoparticles on enhancement of cellular uptake, cellular accumulation of methylene blue in nanoparticles was compared to that in solution. In MCF-7 cells, nanoparticles resulted in significantly (P<0.05) higher cellular accumulation of methylene blue than that in solution. Treatment with methylene blue in nanoparticles resulted in 2-fold higher accumulation of the drug than that in solution.

### Nanoparticle-Mediated Ex Vitro ROS Production

To study the effect of encapsulation in nanoparticles on ROS yield, the amount of ROS generated after photo-activation of methylene blue in nanoparticles was compared to that in solution. ROS generated after light-activation of methylene blue resulted in the generation of reactive oxygen species which convert of APF to fluorescein and increase in fluorescence. Encapsulation of methylene blue in nanoparticles resulted in significantly (P<0.05, ANOVA) higher fluorescence which indicated higher ROS yield with nanoparticles-encapsulated methylene blue. To evaluate the effect of dose of methylene blue on ROS yield, fluorescence was measured after light-activation with two different doses of methylene blue (0.3 or 0.6 μM). At 0.6 μM concentration, photo-activation of methylene blue was more significant and showed 2-fold increase in fluorescence compared to 0.3 μM which indicated increased ROS yield. PBS and Empty nanoparticles treated with equivalent dose of light showed negligible amount of ROS yield.

In order to study the effect of inactive components of nanoparticles on the production of ROS, empty nanoparticles and methylene blue in solution of equivalent concentrations to that used in MB-loaded nanoparticles, were mixed and treated as above. Measured fluorescence of the mixture showed no significant increase in the ROS yield compared to methylene blue in solution. This indicated that the presence of methylene blue unassociated with nanoparticles was not enough for generation of ROS and that methylene blue should be in close proximity to the nanoparticles.

To study the effect of dose of light on the ROS yield, methylene blue in nanoparticles or in solution was photo-activated as described above with 10 consecutive doses of light (1200 mJ/cm² per dose). Increased fluorescence after each illumination indicated increased production of ROS.

This Example indicates that encapsulation of methylene blue in AOT-alginate nanoparticles enhanced its photodynamic cytotoxicity in vitro. AOT-alginate nanoparticles are an ideal carrier system to deliver MB and enhance its PDT.

### Example 5

### Surfactant-Polymer Nanoparticles Overcome P-Glycoprotein-Mediated Drug Efflux

This Example was performed to evaluate the drug delivery potential of AOT-alginate nanoparticles in drug resistant cells overexpressing the drug efflux transporter, P-glycoprotein (P-gp). AOT-alginate nanoparticles were formulated using an emulsion-cross-linking process. Rhodamine 123 and doxorubicin were used as model P-gp substrates. Cytotoxicity of nanoparticle-encapsulated doxorubicin and kinetics of nanoparticle-mediated cellular drug delivery were evaluated in both drug-sensitive and -resistant cell lines.

A surfactant-polymer nanoparticle system was used. These nanoparticles were formulated using dioctylsodium sulfosuccinate [Aerosol OT (AOT)] and sodium alginate. AOT is an anionic surfactant that is approved by the U.S. Food and Drug Administration as oral, topical, and intramuscular excipient. Sodium alginate is a naturally occurring polysaccharide polymer that has been extensively investigated for drug delivery and tissue engineering applications. (Iskakov, R. M. et al. J. Controlled Release 2002, 80:57-68; Shimizu, T. et al. Biomaterials 2003, 24:2309-2316.) This Example demonstrates that AOT-alginate nanoparticles overcame P-gp-mediated drug efflux and drug resistance in P-gp-overexpressing cells without the use of additional P-gp inhibitors.

Rhodamine 123, doxorubicin, sodium alginate, polyvinyl alcohol, and calcium chloride were obtained from Sigma-Aldrich (St. Louis, Mo.). AOT, methanol, and methylene chloride were obtained from Fisher Scientific (Chicago, Ill.).

Nanoparticles were formulated as follows. An aqueous solution of sodium alginate [1.0% (w/v), 1 mL] and drug (5 mg) was emulsified into an AOT solution in methylene chloride [5% (w/v), 2 mL] using sonication over an ice bath. The primary emulsion was further emulsified into 15 mL of a 2% (w/v) aqueous PVA solution by sonication for 1 min to form a water-in-oil-in-water emulsion. Five milliliters of an aqueous calcium chloride solution [60% (w/v)] was added to the emulsion described above with stirring. The emulsion was stirred over night to evaporate methylene chloride. Nanoparticles formed were recovered by ultracentrifugation (Beckman, Palo Alto, Calif.) at 145000 g, washed two times with distilled water to remove unentrapped drug, resuspended in water, and lyophilized. Drug loading in nanoparticles was assessed by extracting 5 mg of nanoparticles with 5 mL of methanol for 30 min and analyzing the methanol extract for drug content. Doxorubicin and rhodamine concentrations were determined by HPLC (see below). Drug loading was represented as percent (w/w) and defined as the amount of drug encapsulated in 100 mg of nanoparticles. Particle size and ξ potential were determined using the Brookhaven 90Plus ξ potential equipment fitted with particle sizing software (Brookhaven Instruments, Holtsville, N.Y.). Nanoparticles (0.1 mg) were dispersed in 1 mL of distilled water by sonication and were subjected to both particle size and ξ potential analysis.

For HPLC determination of doxorubicin and rhodamine, a Beckman Coulter HPLC system connected to Linear Fluor LC 305 fluorescence detector (Altech) and a C-18 column (Beckman Ultrasphere, octadecylsilane, 4.6 mm×250 mm) were used. For doxorubicin, a 70:30 acetonitrile/water (adjusted to pH 3 with glacial acetic acid) mixture was used as the mobile phase at a flow rate of 1 mL/min. For rhodamine, a 50:20:30 acetonitrile/sodium acetate (adjusted to pH 4 with glacial acetic acid)/tetrabutylammonium bromide mixture was used as the mobile phase at a flow rate of 1 mL/min. Detection wavelengths were 505 and 550 nm for doxorubicin and 490 and 526 nm for rhodamine. Retention times were 7 and 3.2 min for doxorubicin and rhodamine, respectively.

Human breast cancer cells (MCF-7) and RPMI-1640 medium were obtained from American Type Culture Collection (ATCC, Manassas, Va.). NCI-ADR/RES (previously known as MCF-7/ADR) cells were obtained from the National Cancer Institute. Both cell lines were passaged in T-75 tissue culture flasks in RPMI-1640 medium supplemented with 10% (v/v) fetal bovine serum.

For cytotoxicity studies, NCI-ADR/RES or MCF-7 cells were seeded in 96-well plates at a seeding density of 5000-10000 cells per well per 0.1 mL of medium and allowed to attach overnight. Following attachment, cells were treated with doxorubicin in solution or doxorubicin in nanoparticles. Untreated cells and empty nanoparticles were used as controls. The medium was replaced every alternate day, and no further dose of doxorubicin or nanoparticles was added. Cytotoxicity was determined over a period of 10 days using a commercially available MTS assay (Promega). Results were analyzed by using an ANOVA. Differences were considered significant at P<0.05.

For uptake studies, nanoparticles containing rhodamine 123 were used for the study to avoid the complications of doxorubicin-induced cytotoxicity while evaluating drug accumulation. All the studies were performed at 37° C. unless specified. Cells were seeded in a 24-well plate at a density of 50,000 cells/well and allowed to attach overnight. Following attachment, cells were treated with rhodamine in solution or encapsulated in nanoparticles. To determine the effect of the dose of nanoparticles on rhodamine uptake, cells were treated with various doses (25-300 μg/mL) of nanoparticles containing rhodamine for 2 h. To determine the effect of ATP depletion on nanoparticle uptake, cells were preincubated with growth medium containing 0.1% (w/v) sodium azide and 50 mM deoxyglucose for 1 h and then incubated with a nanoparticle suspension (100 μg/mL) containing 0.1% (w/v) sodium azide and 50 mM deoxyglucose for 2 h.

To study the effect of inhibition of active processes on cellular uptake of nanoparticles, cells were preincubated at 4° C. for 1 h and then treated with the nanoparticle suspension (100 μg/mL) at 4° C. for 2 h. To determine the effect of blank nanoparticles on rhodamine uptake, cells were treated with a mixture of blank nanoparticles (0, 30, or 300 μg/mL) and rhodamine in solution. To determine the effect of blank nanoparticles on fluorescein uptake, cells were treated with a mixture of blank nanoparticles (30 or 300 μg/mL) and fluorescein in solution.

At the end of the treatment period, cells were washed three times with cold PBS and then lysed using 100 μL of cell culture lysis reagent (CCLR; Promega). The protein content of the cell lysates was determined using the Pierce (Rockford, Ill.) BCA protein assay. Cell lysates were then mixed with 300 μL of methanol and incubated at 37° C. for 6 h at 100 rpm. Samples were centrifuged at 14 000 rpm for 10 min at 4° C. The concentration of rhodamine in the methanolic extract was determined by HPLC as described before. Data were expressed as rhodamine accumulation normalized to total cell protein. For fluorescence microscopy, the uptake and intracellular distribution of doxorubicin in NCI-ADR/RES cells were determined qualitatively using fluorescence microscopy. Cells (5×105) were seeded on coverslips placed in 35 mm dishes.

The following day, medium was replaced with fresh medium containing 2.5 μg/mL doxorubicin in solution or in nanoparticles. At 2 h post-treatment, cells were rinsed with drug-free medium and incubated with 75 nM Lysotracker Green (Invitrogen) for 30 min. Cells were then washed and counterstained with DAPI (4′,6-diamidino-2-phenylindole, Invitrogen). Images were captured with a BX60 Olympus fluorescence microscope. Images captured using red, blue, and green filters were overlaid to determine localization and association of doxorubicin-associated red fluorescence in the nucleus and endolysosomes, respectively.

The following results were obtained from the experiments described above.

AOT-Alginate Nanoparticles Loaded with Doxorubicin or Rhodamine.

Nanoparticles used in the Example were essentially similar to those reported. (Chavanpatil, M. D. et al., Pharm. Res. 2007, 24:803-810) Both rhodamine-loaded nanoparticles and doxorubicin-loaded nanoparticles were in a similar size range (500-700 nm) and had similar polydispersity indices (˜0.28). The ξ potential of nanoparticles containing doxorubicin or rhodamine was around −13 to −14 mV. It was expected that the ξ potential reported for these formulations would be marginally stable. Drug loading was 4.6% (w/w) and 3.8% (w/w) for rhodamine and doxorubicin, respectively. The suspension stability of nanoparticles was unaffected by lyophilization, salt, or the presence of serum.

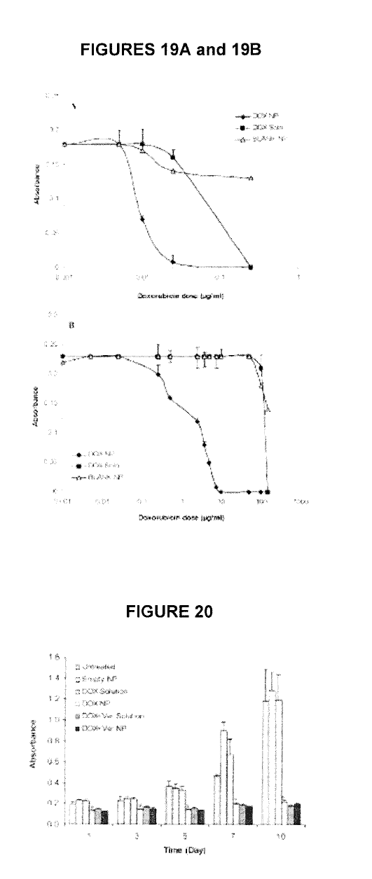

Enhanced and Sustained Cytotoxicity in MDR Cells. The cytotoxicity of nanoparticle-encapsulated doxorubicin was evaluated in vitro. Drug-sensitive MCF-7 cells demonstrated dose-dependent cytotoxicity to doxorubicin in solution, whereas concentrations of >50 μg/mL were required to induce cytotoxicity in the drug-resistant NCI-ADR/RES cells (FIG. 19A,B). Addition of verapamil, a P-gp inhibitor, reversed the resistance to doxorubicin in NCI-ADR/RES cells (FIG. 20). Nanoparticles enhanced the cytotoxicity of doxorubicin significantly in both drug-sensitive and drug-resistant cells. Nanoparticle-mediated enhancement of cytotoxicity observed in the drug-resistant cells was sustained during the 10 days of the study [P<0.05 for all the days that were tested (FIG. 20)]. There was no additional benefit of combining verapamil with doxorubicin in nanoparticles. Blank nanoparticles had no effect on cell survival, indicating that blank nanoparticles were not toxic to cells in the dose range that was tested.

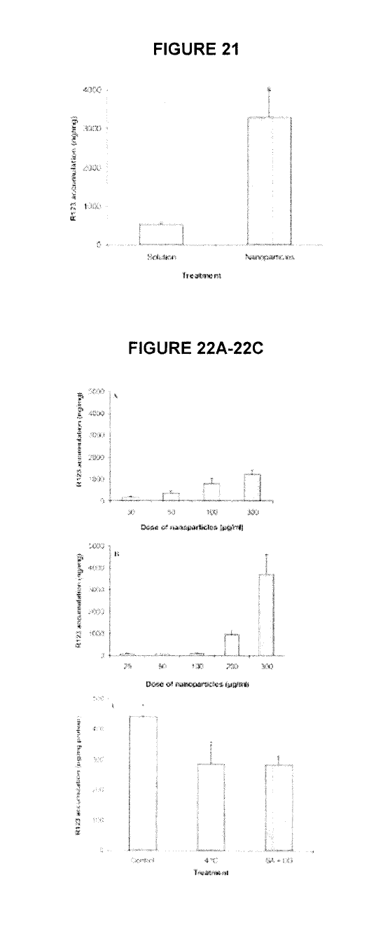

Kinetics of Accumulation of Rhodamine in Resistant and Sensitive Cells. To determine the efficacy of cellular drug delivery with AOT-alginate nanoparticles, cellular accumulation of rhodamine, a P-gp substrate, following treatment with an equivalent dose of rhodamine was compared in solution and in nanoparticles. As shown in FIG. 21, treatment with rhodamine in nanoparticles resulted in a significantly higher level of accumulation of rhodamine than treatment with rhodamine in solution (P<0.05).

To determine the kinetics of drug accumulation with nanoparticles, the cellular accumulation of rhodamine was evaluated following treatment with different doses of nanoparticles containing rhodamine. As shown in FIG. 22A, in drug sensitive MCF-7 cells, the level of accumulation of rhodamine increased in proportion to the nanoparticle dose. However, in drug-resistant NCI-ADR/RES cells, the level of rhodamine accumulation was low and nonlinear at nanoparticle doses of less than 100 μg/mL (FIG. 22B). At doses above 200 μg/mL, nanoparticles significantly enhanced cellular accumulation of rhodamine.

To determine the mechanism of uptake of nanoparticles into cells, the energy dependence of nanoparticle uptake in cells was evaluated. Decreasing the rate of endocytosis by incubating cells at 4° C. or with metabolic inhibitors resulted in an ˜40% reduction in the rate of cellular uptake of nanoparticles (FIG. 22C). The energy dependence of nanoparticle uptake, along with dose and time dependence, suggests that cells internalize AOT-alginate nanoparticles through an endocytic process.

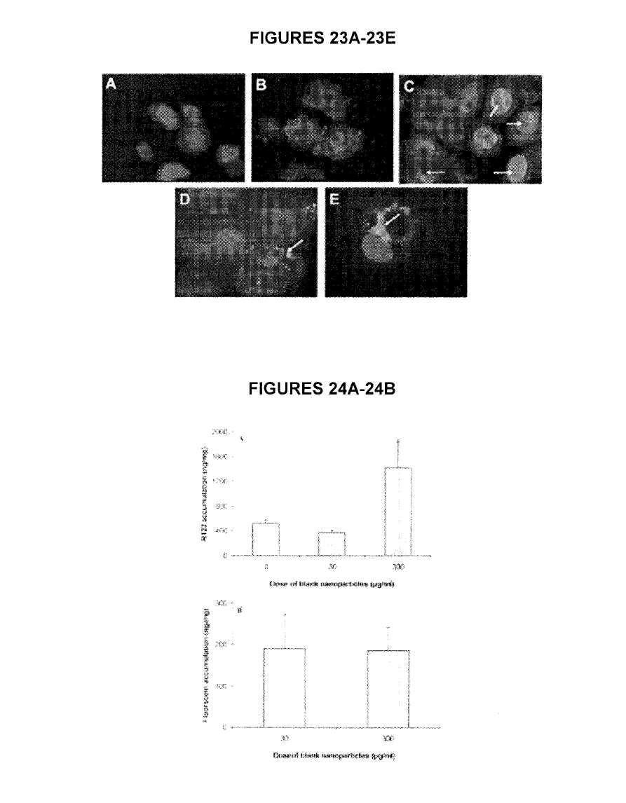

Intracellular Distribution of Doxorubicin. To determine whether encapsulation of doxorubicin in nanoparticles affected its trafficking inside drug-resistant cells, the intracellular distribution of free and nanoparticle-encapsulated doxorubicin was evaluated in NCI-ADR/RES cells that were stained for nucleus and endolysosomes. Free doxorubicin demonstrated a diffuse distribution within the cells, with a significant fraction appearing in vesicles located near the cell membrane (FIG. 23B,D). Those vesicles stained positively with Lysotracker Green (FIG. 23D), indicating that they were endolysosomal in nature. A significant proportion of nanoparticle-encapsulated doxorubicin also appeared to be present in endolysosomal vesicles (FIG. 23C,E); however, these vesicles were concentrated at the peri-nuclear region rather than at the cell periphery. Further, doxorubicin was also present in the nuclei of cells treated with nanoparticle-encapsulated doxorubicin (FIG. 23C). No or insignificant doxorubicin fluorescence was observed in nuclei of cells treated with doxorubicin in solution (FIG. 23B).

Effect of Blank Nanoparticles on Rhodamine and Fluorescein Uptake. To determine whether blank nanoparticles had any effect on drug efflux, the accumulation of rhodamine in drug-resistant cells was studied in the presence and absence of blank nanoparticles. As shown in FIG. 24A, blank nanoparticles significantly enhanced rhodamine accumulation in drug-resistant cells at a nanoparticle dose of 300 μg/mL (P<0.05) but not at a dose of 30 μg/mL. To determine whether nanoparticle-mediated enhancement in cellular uptake was nonspecific, the effect of blank nanoparticles was evaluated on the cellular accumulation of fluorescein sodium in drug-resistant cells. As shown in FIG. 24B, irrespective of the nanoparticle dose, blank nanoparticles did not affect the cellular accumulation of fluorescein.

The objective of this Example was to determine whether doxorubicin, a P-gp substrate, encapsulated in AOT-alginate nanoparticles was susceptible to P-gp-mediated drug efflux. Cytotoxicity studies in P-gp-overexpressing tumor cells demonstrated that nanoparticles loaded with doxorubicin alone were as effective as nanoparticles containing both doxorubicin and verapamil, suggesting that AOT-alginate nanoparticles can overcome P-gp-mediated drug resistance. However, this effect was dose-dependent; enhanced cytotoxicity was observed with a 300 μg/mL dose of nanoparticles but not with a 30 μg/mL. Sustained cytotoxicity observed with nanoparticle-encapsulated doxorubicin correlates well with the sustained release properties of AOT-alginate nanoparticles. The inventors previously showed that AOT-alginate nanoparticles sustain the release of encapsulated doxorubicin over 15 days. (Chavanpatil, M. et al., Polymer-surfactant nanoparticles for sustained release of water-soluble drugs. J. Pharm. Sci. 2006, in press.) Further, doxorubicin-loaded nanoparticles resulted in sustained cytotoxicity in drug-sensitive MCF-7 cells over 10 days of the study. (Chavanpatil, M. D. et al., Pharm. Res. 24:803-810, 2007)

Thus, the duration of cytotoxicity observed in drug-resistant cells in this Example is similar to that observed in drug-sensitive cells in the inventors' previous study. The inventors showed that an increased level of cellular drug accumulation following treatment with AOT-alginate nanoparticles contributes to the enhanced therapeutic efficacy of a nanoparticle-encapsulated drug in drug-sensitive cells as well. (Chavanpatil, M. D. et al., Pharm. Res. 24:803-810, 2007) To evaluate whether AOT-alginate nanoparticles increase the level of drug accumulation in drug resistant cells, the cellular accumulation of rhodamine, another model P-gp substrate, was assessed in NCI/ADR-RES cells. The results showed that cells treated with nanoparticle-encapsulated rhodamine demonstrated higher levels of accumulation of rhodamine than those treated with a rhodamine solution.

To further understand the dose effect observed in cytotoxicity studies, the dose response in cellular accumulation of rhodamine was determined in both drug-sensitive and -resistant cells. In drug-sensitive cells, nanoparticles demonstrated a near-linear dose-response relationship. A similar dose-response relationship has been observed for other nanoparticle systems and in other cell types that do not overexpress P-gp. (Chavanpatil, M. D. et al., Pharm. Res. 24:803-810, 2007.) However, in drug-resistant cells, an inflection was observed in the dose-response curve, with significant drug accumulation observed only at doses higher than 200 μg/mL. This is consistent with the observations that nanoparticles enhanced doxorubicin cytotoxicity in P-gp overexpressing cells at a 300 μg/mL dose but not at a 30 μg/mL dose. Previous studies have shown that certain excipients such as Pluronics and polyethylene glycol can inhibit P-gp mediated drug efflux. (Batrakova, E. V. et al., Br. J. Cancer 2001, 85, 1987-1997; Shen, Q. et al., Int. J. Pharm. 2006, 313:49-56.)

To determine whether AOT-alginate nanoparticle formulation has a similar activity, the effect of blank nanoparticles on the cellular accumulation of rhodamine was investigated. Because the reversal of drug efflux appeared to be dependent on nanoparticle dose, two doses were used in the study. Consistent with the previous finding, enhancement in cellular accumulation was observed with the 300 μg/mL blank nanoparticle dose and not the 30 μg/mL dose.

One possible mechanism by which nanoparticles could enhance cellular accumulation of P-gp substrates is through permeabilization of the cell membrane. This is especially a concern, because surfactants are known to create pores in cellular membranes (Bogman, K. et al., J. Pharm. Sci. 2003, 92:1250-1261). and nanoparticles used in this study contain anionic surfactant AOT. If the increased level of cellular accumulation observed with nanoparticles in this study were attributable to a permeabilized cell membrane, then it would be expected that similar enhancements would be seen in cells without P-gp overexpression and with drugs that are not P-gp substrates and that nanoparticles would cause toxicity. Blank nanoparticles, at the 300 μg/mL dose, did not enhance the accumulation of rhodamine in the non-P-gp-expressing MCF-7 cells. Similarly, blank nanoparticles did not enhance the accumulation of fluorescein sodium, a non-P-gp substrate, in P-gp-overexpressing cells. Further, blank nanoparticles did not cause a significant toxicity in NCI/ADR-RES cells at the 300 μg/mL dose. The energy dependence of nanoparticle accumulation in cells suggests the involvement of endocytosis in nanoparticle uptake. Taken together, these results provide compelling evidence that the effects of the nanoparticles on drug or probe accumulation are not due to nonspecific effects on membrane permeabilization.

To further understand the mechanism of efficacy of nanoparticle-encapsulated doxorubicin, the intracellular trafficking of doxorubicin was studied. Doxorubicin causes cytotoxicity in tumor cells through several mechanisms; however, intercalation with genomic DNA in the nucleus and topoisomerase inhibition are considered primary events in doxorubicin-induced cytotoxicity. Thus, the nucleus is the chief site of action for doxorubicin. Interestingly, cells treated with nanoparticle-encapsulated doxorubicin were found to accumulate doxorubicin in the nucleus, whereas cells treated with a doxorubicin solution did not. Enhanced nuclear delivery of doxorubicin by AOT-alginate nanoparticles could have contributed to the enhanced cytotoxicity observed with nanoparticle-encapsulated doxorubicin. Enhanced nuclear accumulation of doxorubicin could be explained on the basis of the increased level of cellular accumulation of doxorubicin due to inhibition of P-gp-mediated drug efflux. The fact that blank nanoparticles also enable an increased level of cellular accumulation of free doxorubicin supports this hypothesis.

In addition to P-gp inhibition, another significant advantage of AOT-alginate nanoparticles is the fact that following encapsulation of weakly basic drugs, nanoparticles have a net negative charge, which stabilizes nanoparticles in buffer and in medium containing serum. This is an advantage over other nanoparticle delivery systems such as polycyanoacrylate nanoparticles that become cationic following encapsulation of weakly basic drugs like doxorubicin. (Bogman, K. et al., J. Pharm. Sci. 2003, 92, 1250-1261.) Due to the presence of an excess of highly electronegative sulfosuccinate groups from AOT and carboxyl groups from alginate in nanoparticles, loading of cationic drugs is not believed to alter the potential of nanoparticles. The ability to sustain doxorubicin-induced cytotoxicity over a period of 10 days is another important advantage of AOT-alginate nanoparticles over other delivery systems.

Example 5 therefore demonstrates that encapsulation of doxorubicin in AOT-alginate nanoparticles resulted in a significant and sustained enhancement of doxorubicin-induced cytotoxicity in drug-resistant tumor cells. Increased therapeutic efficacy of nanoparticle-encapsulated drug was associated with an increase in the level of cellular and nuclear drug accumulation. An increase in the level of cellular accumulation was observed even with a mixture of blank nanoparticles and rhodamine solution. Enhancement of cellular accumulation of rhodamine in drug-resistant cells was not caused by membrane permeabilization.

While the description above refers to particular embodiments of the present invention, it will be understood that many modifications may be made without departing from the spirit thereof. The presently disclosed embodiments are therefore to be considered in all respects as illustrative and not restrictive. All patents and publications referenced are incorporated herein by reference.

1. A nanoparticle composition comprising alginate, aerosol OT, and a therapeutic agent.

2. The nanoparticle composition of claim 1, wherein said therapeutic agent is a cancer therapeutic agent.

3. The nanoparticle composition of claim 1, wherein said therapeutic agent is a therapeutic agent effective for treating psoriasis.

4. The nanoparticle composition of claim 2, wherein said therapeutic agent is selected from the group consisting of doxorubicin, verapamil, and clonidine.

5. The nanoparticle composition of claims 3, wherein the therapeutic agent is selected from the group consisting of Anthralin, Dovonex, Taclonex, Tazorac, topical steroid, and salicylic acid.

6. A method of treating a proliferative disease in an individual, comprising administering to the individual a nanoparticle composition of claim 1.

7. The method of claim 6, wherein the therapeutic agent inhibits cell proliferation.

8. The method of claim 6, wherein the proliferative disease is cancer.

9. The method of claim 6, wherein the average diameter of the nanoparticles in the composition is between 10 and 1000 nanometers.

10. The method of claim 6, wherein the average diameter of the nanoparticles in the composition is between 30 and 500 nanometers.

11. The method of claim 6, wherein the average diameter of the nanoparticles in the composition is between 50 and 350 nanometers.

12. The method of claim 6, wherein the therapeutic agent is selected from the group consisting of doxorubicin, verapamil, and cholodine.

13. The method of claim 6, wherein the individual is human.

14. A method of treating a skin disorder is an individual, comprising administering to the individual a composition comprising nanoparticles comprising alginate and aerosol OT, wherein said nanoparticles further comprise an amount of at least one therapeutic agent.

15. The method of claim 14, wherein said skin disorder is psoriasis.

16. The method of claim 14, wherein said therapeutic agent is selected from the group consisting of Anthralin, Dovonex, Taclonex, Tazorac, topical steroid, and salicylic acid.

17. The method of claim 16, wherein the average diameter of the nanoparticles in the composition is between 30 and 500 nanometers.

18-26. (canceled)

27. A method for treating psoriasis in an individual comprising administering to the individual a composition comprising nanoparticles comprising alginate and aerosol OT, wherein said nanoparticles further comprise an amount of at least one therapeutic agent selected from the group consisting of Methotrexate, cyclosporine, and a steroid.
